(19) United States
(12) Patent Application Publication
(10) Pub. No.: US 2010/0016387 A1
(43) Pub. Date: Jan. 21, 2010

(54) METHOD OF TREATMENT

(76) Inventors: Avirup Bose, Marlborough, MA (US); Thomas Edward Hughes, Concord, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/518,551

(22) PCT Filed: Dec. 10, 2007
(86) PCT No.: PCT/US2007/086919

§ 371 Date: Jun. 10, 2009

Related U.S. Application Data
(60) Provisional application No. 60/869,448, filed on Dec. 11, 2006.

(51) Int. Cl. A61K 31/428; A61K 31/423; A61K 31/4184; A61K 31/403

(52) U.S. Cl. 514/367; 514/375; 514/394; 514/417

(57) ABSTRACT

Method of preventing or treating myocardial ischemia by inhibiting DGAT1 enzyme with a DGAT1 inhibitor compound.

### FIELD OF INVENTION

Method of preventing or treating myocardial ischemia by inhibiting DGAT1 enzyme with a DGAT1 inhibitor.

### BACKGROUND OF THE INVENTION

The mechanical power of the myocardium is supported by high rates of oxygen consumption and the metabolism of carbon fuels, fatty acids and carbohydrates. Fatty acids are the main fuel for the adult heart, supplying almost 60-80% of the energy and the balance comes from oxidation of glucose and lactate. The ATP is broken down to fuel contractile work and is resynthesized in the mitochondria from the oxidation of fatty acids, glucose and lactate. Myocardial ischemia is a metabolic disease which occurs when the coronary blood flow is insufficient to supply enough oxygen to combust carbon fuels and resynthesize ATP at the normal rate. This results in an increase in glucose uptake by the heart to fuel glycolysis. However, unlike aerobic conditions, this glucose is not readily oxidized in the mitochondria but rather converted to lactate. As a result, the heart which normally takes up lactate switches to production of lactate. This results in a drop in intracellular pH and ATP and a decrease in contractile work. Paradoxically, the ischemic heart continues to derive most of its energy from fat oxidation (50-70%) despite a high rate of lactate production. The high ratio of NADH/NAD+ and acetyl CoA/free CoA resulting from the β-oxidation inhibits pyruvate dehydrogenase and continues to inhibit the oxidation of pyruvate (FIG. 1).

Ischemia-induced disruption of cardiac metabolism can be minimized by decreasing fatty acid oxidation and increasing the rates of glucose and lactate combustion [Stanley, W. C., Expert opinion in Investig. Drugs; 11(5): 615-629, 2002]. Proof of this approach is evidenced by the success of the anti-anginal drug trimetazidine. Trimetazidine does not trigger any direct effect on heart rate or cardiac contractility nor lower blood pressure like traditional hemodynamic agents. It exerts its effects through partial inhibition of fatty acid oxidation and has been shown to partially decrease the decline in tissue pH and improve contractile function during low-flow ischemia in isolated perfused rat hearts. Further evidence of the above mentioned principle was recently obtained from studies involving Malonyl CoA decarboxylase inhibitors in ex vivo working rat hearts. Malonyl CoA decarboxylase (MCD) converts malonyl CoA to Acetyl CoA and thereby removes the inhibitory effect of malonyl CoA on fatty acid oxidation. Using inhibitors to MCD the authors reported a decrease in fatty acid oxidation and an increase in glucose oxidation in the ex vivo working rat hearts (Dyck, J. R. and Lopaschuk, G. D., J. Mol. Cell. Cardiol. 34(9): 1099-1109, 2002).

Current therapies for myocardial ischemia includes delivering more oxygen to the heart via coronary vasodilation or by decreasing the need for ATP by reducing heart rate and/or arterial blood pressure, and through intravenous infusion of glucose, insulin, and potassium. An alternative approach that has been suggested involves partial inhibition of fatty acid oxidation or by blocking fatty acid entry into the mitochondria which might result in an increase of pyruvate oxidation.

### SUMMARY OF THE INVENTION

The present invention discloses that inhibition of DGAT1 activity in cardiomyocytes inhibits fatty acid oxidation. Moreover, in presence of glucose, the inhibition in fatty acid oxidation is more profound indicating a substrate switch from fatty acids to glucose for ATP generation.

DGAT is an enzyme that catalyzes the last step in triacylglycerol biosynthesis. DGAT catalyzes the coupling of a 1,2-diacylglycerol with a fatty acyl-CoA resulting in Coenzyme A and triacylglycerol. Two enzymes that display DGAT activity have been identified: DGAT1 (acyl coA-diacylglycerol acyl transferase 1, see Cases et al, Proc. Natl. Acad. Sci. 95:13018-13023, 1998) and DGAT2 (acyl coA-diacylglycerol acyl transferase 2, see Cases et al, J. Biol. Chem. 276:38870-38876, 2001). DGAT1 and DGAT2 do not share significant protein sequence homology. Importantly, DGAT1 knockout mice are protected from high fat diet-induced weight gain and insulin resistance (Smith et al, Nature Genetics 25:87-90, 2000). The phenotype of the DGAT1 knockout mice suggests that a DGAT1 inhibitor has utility for the treatment of obesity and obesity-associated complications.

The following is a non-exhaustive list of patents and patent applications that describe DGAT1 inhibitors. WO0204682: Polymorphisms In A Diacylglycerol Acyltransferase Gene, And Methods Of Use Thereof; WO9745439: DNA Encoding Acylcoenzyme A: Cholesterol Acyltransferase And Uses Thereof; US20030154504: Methods And Compositions For Modulating Carbohydrate Metabolism; US20030167483: Diacylglycerol O-acyltransferase; WO9967403: Diacylglycerol O-acyltransferase; WO9967268: Diacylglycerol O-acyltransferase; WO05013907: Pyrrolo[1,2-b]pyridazine Derivatives; WO05044250: Use Of Sulfonamide Compounds For The Treatment Of Diabetes And/or Obesity; WO06064189: Oxadiazole Derivatives As DGAT Inhibitors; WO06004200: Urea Derivative; WO06019020: Substituted Ureas; US20040209838: Modulation Of Diacylglycerol Acyltransferase 1 Expression; US20040185559: Modulation Of Diacylglycerol Acyltransferase 1 Expression; WO04047755: Fused Bicyclic Nitrogen-containing Heterocycles; US20040224997: Preparation And Use Of Aryl Alkyl Acid Derivatives For The Treatment Of Obesity; WO05072740: Anorectic Compounds; JP2006045209: Urea Derivative; WO06044775: Preparation And Use Of Biphenyl-4-yl-carbonylamino Acid Derivatives For The Treatment Of Obesity; JP2004067635: DGAT Inhibitor; JP2005206492: Sulfonamide Compound; and U.S. Pat. No. 6,100,077: Isolation Of A Gene Encoding Diacylglycerol Acyltransferase.

Inventors incorporate the above references by their entirety in regards to the disclosure of DGAT1 inhibitor compounds.

### BRIEF DESCRIPTION OF THE FIGURES

FIG. 1. Schematic depiction of pyruvate oxidation during demand-induced ischemia. Reprinted from William C. Stanley, Expert Opin Investig Drugs (2002), 11(5), p 615-629

FIG. 2. Effect of DGAT1 inhibition on fatty acid oxidation in neonatal cardiomyocytes in absence of glucose

FIG. 3. Effect of DGAT1 inhibition on fatty acid oxidation in neonatal cardiomyocytes in presence of glucose

### DETAILED DESCRIPTION OF THE INVENTION

Myocardial ischemia is characterized by reduced formation of ATP by aerobic mechanisms resulting in an accelerated rate of glycolysis and accumulation of lactate. Decrease of intracellular pH resulting from lactate accumulation lead to less contractile work and poor ion homeostasis. Partial inhibition of fatty acid oxidation and/or increased pyruvate oxidation will lower lactate concentrations and reduce metabolic abnormalities associated with myocardial ischemia. The present invention discloses that inhibition of DGAT1 in primary rat cardiomyocytes inhibits fat oxidation and switches substrate utilization to glucose to generate ATP. Inhibition of DGAT1 activity in rat cardiomyocytes inhibits fatty acid oxidation. Moreover, in presence of glucose, DGAT1 inhibition switches substrate oxidation from fatty acids to glucose. The inhibition of DGAT1 activity will therefore be therapeutically beneficial for treatment of myocardial ischemia. Thus, orally active or parenterally administered DGAT1 inhibitors provide a novel therapeutic approach for the treatment of myocardial ischemia.

The current discovery thus implies that DGAT1 inhibition in cardiac muscle will decrease fatty acid oxidation and increase glucose oxidation and thus provide the basis for therapeutic intervention of myocardial ischemia.

DGAT1 knockout in mice leads to an increase in whole body energy expenditure. This result suggested that DGAT1 inhibition might lead to increased fatty acid oxidation in muscle. However, our results in cardiomyocytes demonstrate that DGAT1 inhibition in these cells has the opposite effect.

### Methods

### Preparation and Maintenance of Neonatal Rat Primary Cardiomyocytes

Neonatal Rat Ventricular Myocytes (NRVM's) were extracted from 1-3 day old Sprague Dawley rat pups. The atria was removed and discarded. Both the right and left ventricles were digested in CBHHF media containing 0.2% Trypsin, 100 U Penn-Strep and DNAse II. DNase II was added to the media to decrease the viscosity due to cell rupture. Fibroblasts were separated from the myocytes by preplating for 30 min. Fibroblasts firmly adhered to tissue culture plates leaving myocytes in suspension. Myocytes were then collected and cultured in MEM/5% FBS/Pen-Strep/BrDU/L-Gln overnight at 37 C, 5.0% CO2 at a confluency of 75-80% (1.8×10⁶ cells per well of a 6 well plate and 0.9×10⁶ cells per well of a 12 well plate). The following day myocyte containing plates were assayed for fatty acid oxidation capacity.

Fatty Acid Oxidation of Rat Primary Cardiomyocytes with DGAT1 inhibitors.

{4-[4-(4-Amino-7,7-dimethyl-7H-pyrimido[4,5-b][1,4]oxazin-6-yl)-phenyl]-cyclohexyl}-acetic acid is a representative known DGAT1 inhibitor. This compound is disclosed in WO 2004/047755.

Rat primary cardiomyocytes were plated in either 6 well plate or 12 well plate. The cells were treated with {4-[4-(4-Amino-7,7-dimethyl-7H-pyrimido[4,5-b][1,4]oxazin-6-yl)-phenyl]-cyclohexyl}-acetic acid at a final concentration of 1 μM or with DMSO control for two hours. The cells were then washed once with PBS and were incubated in 2 ml non-bicarbonate assay buffer [114 mM of NaCl, 4.7 mM of KCl, 1.2 mM of KH₂PO₄, 1.2 mM of MgSO₄, and 0.5% fatty acid free BSA (Sigma Cat# A0281), ¹⁴C-palmitate (American Radiolabeled Chemicals Inc., 50-60 mCi/mmol, 0.5 mCi/ml, Cat# ARC-172A) at a final concentration of 36 μM with or without 0.5 mM glucose for 2 hours. ¹⁴CO₂ released by the cells were measured as described below. Briefly, seventy two hours after transfection, culture medium was removed and cells were washed once with PBS and then 2 ml non-bicarbonate assay buffer [114 mM of NaCl, 4.7 mM of KCl, 1.2 mM of KH2PO₄, 1.2 mM of MgSO₄, and 0.5% fatty acid free BSA (Sigma Cat# A0281) was added into each well. then the cells were labeled with ¹⁴C-palmitate (American Radiolabeled Chemicals Inc., 50-60 mCi/mmol, 0.5 mCi/ml, Cat# ARC-172A) at a final concentration of 50 μM for 2 hours. After labeling, assay buffer was transferred into a 15 ml Falcon tube with a stopper top (Fisher Cat # K882310-0000), in which a center well (Fisher Cat# K882320-0000) was attached. Inside of the center well, a paper fan made of a piece of Whatman filter paper #1 (Fisher Cat# 09-805G) at 1 inch×1.5 inch and soaked with 250 μl of 2N NaOH was placed. Immediately, 1.5 ml of 6N HCl was injected into the tube by a 3 cc syringe and then allowed to stand overnight. 1 ml of H₂O and 62 μl of 2N NaOH were added to a 20 ml glass scintillation vial (Fisher Cat# 033374) and the filter paper was transferred from the center well to the vial. 10 ml of Aqualsol II (Perkin Elmer Cat# 6NE9529) was added and mixed with the filter by vortexing. The sample was counted on a beta scintillation counter after resting for at least 2 hours. The amount of ¹⁴CO₂ (bicarbonate) was used as an index of fatty acid oxidation. Measurements were corrected by subtracting the background and normalized with protein concentration. The protein concentration was measured using the BCA protein assay kit (Pierce#23225). The results were normalized by total protein from each well and are presented as dpm/mg protein

### Statistical Analysis

The statistical analysis on all data was performed using two-tailed, unpaired Student's t-test. A p-value smaller than 0.05 was considered to be statistically significant.

### Results

Fatty Acid Oxidation in Primary Rat Cardiomyocytes Treated with DGAT1 Inhibitors in Absence of Glucose

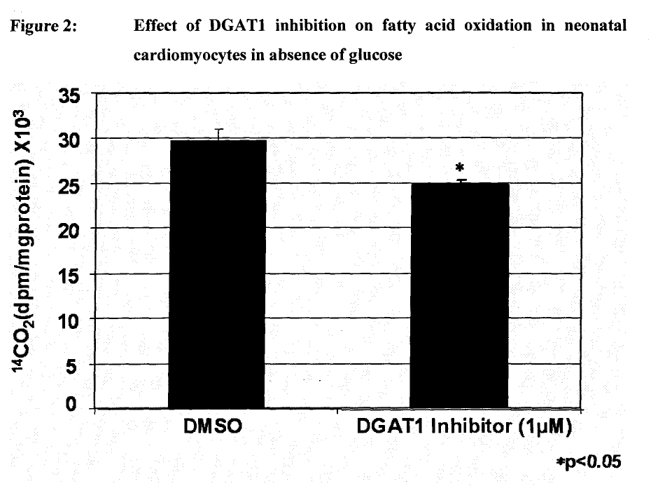

To test whether inhibition of DGAT1 activity had any effect on the fatty acid oxidation capacity in neonatal rat cardiomyocytes we treated these cells with the DGAT1 inhibitor {4-[4-(4-Amino-7,7-dimethyl-7H-pyrimido[4,5-b][1,4]oxazin-6-yl)-phenyl]-cyclohexyl}-acetic acid (1 μM, DGAT1 IC₅₀ ˜0.05 μM). The reaction was carried out in absence of glucose. As the results show (FIG. 2), inhibition of DGAT1 activity with {4-[4-(4-Amino-7,7-dimethyl-7H-pyrimido[4,5-b][1,4]oxazin-6-yl)-phenyl]-cyclohexyl}-acetic acid resulted in a small but significant decrease (˜15%) in the ability of these cells to oxidize [¹⁴C]palmitate.

Primary rat cardiomyocytes were incubated with 1 μM {4-[4-(4-Amino-7,7-dimethyl-7H-pyrimido[4,5-b][1,4]oxazin-6-yl)-phenyl]-cyclohexyl}-acetic acid for 2 hr. The cells were then incubated with ¹⁴C-labeled palmitate and liberated ¹⁴CO₂ was determined as described. N=3, *p<0.05, mean±SEM. The data is representative of the results from more than two independent experiments.

Fatty acid oxidation in primary rat cardiomyocytes treated with {4-[4-(4-Amino-7,7-dimethyl-7H-pyrimido[4,5-b][1,4]oxazin-6-yl)-phenyl]-cyclohexyl}-acetic acid in presence of glucose.

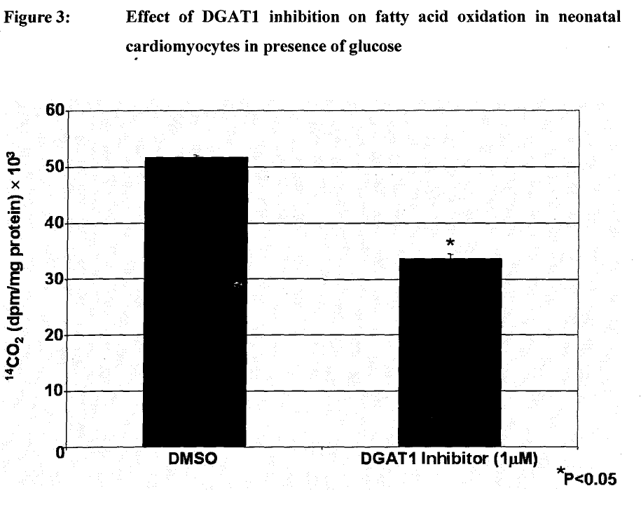

Cardiomyocytes take up glucose via glucose transporters and either stores it as glycogen or metabolizes it via glycolysis to pyruvate. To test whether DGAT1 inhibition will result in switching the energy substrate oxidation away from fatty acids to glucose we tested the ability of {4-[4-(4-Amino-7,7-dimethyl-7H-pyrimido[4,5-b][1,4]oxazin-6-yl)-phenyl]-cyclohexyl}-acetic acid to inhibit fatty acid oxidation in cardiomyocytes in presence of 0.5 mM glucose. The results in FIG. 3 show that addition of glucose leads to a robust decrease in fatty acid oxidation in these cells when they were treated with DGAT1 inhibitors.

Primary rat cardiomyocytes were incubated with 1 μM {4-[4-(4-Amino-7,7-dimethyl-7H-pyrimido[4,5-b][1,4]oxazin-6-yl)-phenyl]-cyclohexyl}-acetic acid for 2 hr. The cells were then incubated with ¹⁴C-labeled palmitate, with 0.5 mM glucose, and liberated ¹⁴CO₂ was determined as described. N=3, *p<0.05, mean±SEM. The data is representative of the results from more than two independent experiments.

Myocardial ischemia occurs when the rate of oxygen consumption and aerobic ATP formation is insufficient to meet the required cardiac power for a given heart rate, arterial blood pressure and inotropic state. This results from an impairment of coronary blood flow (30-60% reduction) resulting in insufficient oxygen supply to support ATP production through fatty acid oxidation. Under these conditions glycolysis is rapidly stimulated along with breakdown of tissue glycogen. However, the pyruvate generated through glycolysis is not readily oxidized to generate ATP in the mitochondria but rather reduced to lactate. This accumulation of lactate leads to a decline in the intracellular pH from normal values. At low intracellular pH the Ca²⁺ concentration for a given amount of force generation is increased. Moreover, the amount of ATP required by the Ca²⁺ pump is greater at lower pH and the residual ATP generated is now directed more towards maintaining the Ca²⁺ homeostasis rather than contractile work of the heart.

Current medical therapies for myocardial ischemia include delivering more oxygen to the heart via coronary vasodilation or by decreasing the need for ATP by reducing heart rate and/or arterial blood pressure. An alternative approach could involve reversing the inhibition in pyruvate oxidation either by blocking fatty acid oxidation (e.g. with trimetazidine) or by blocking fatty acid entry into the mitochondria (e.g. with perhexylline or oxfenicine). All these agents have been shown to increase the rate of pyruvate oxidation and lower the rate of lactate production during ischemia or post ischemic reperfusion. It has been previously observed that inhibition of DGAT1 activity in cultured C2C12 myoblasts lead to an inhibition of fatty acid oxidation in these cells.

The present invention discloses that pharmacological inhibition of DGAT1 does indeed lead to a decrease in fatty acid oxidation in these cells. Moreover, in presence of glucose DGAT1 inhibition has a robust inhibitory effect on the fatty acid oxidation capacity of these cells. DGAT1 inhibition leads to a switch in the substrate that these cells utilize to generate ATP. Under normal conditions these cells use the fatty acid as their major substrate for ATP generation but switch to glucose when DGAT1 is inhibited in these cells. It has been observed that inhibition of DGAT1 in differentiated adipocytes lead to an increase glucose uptake in these cells even in absence of insulin. A similar mechanism might be operating here to allow more entry of glucose in the cardiomyocytes thereby switching the substrate flux from fatty acids to glucose. Thus, DGAT1 inhibition is a therapeutic option for inhibiting fatty acid oxidation and lactate production in the myocardium and thus be beneficial for minimizing metabolic abnormalities in myocardial ischemia.

The present invention contemplates DGAT1 inhibitors as a compound present in a pharmaceutical composition. In view of the close relationship between the free compounds, the prodrug derivatives and the compounds in the form of their salts, whenever a compound is referred to in this context, a prodrug derivative and a corresponding salt is also intended, provided such is possible or appropriate under the circumstances.

The compounds, including their salts, can also be obtained in the form of their hydrates, or include other solvents used for their crystallization.

As described herein above, the compounds contemplated of the present invention may be employed for the treatment of myocardial ischemia mediated by DGAT1 activity.

U.S. Provisional Application No. 60/787,859 filed Mar. 31, 2007 is incorporated by reference as to the non-essential subject matter contained therein.

Listed below are definitions of various terms used to describe the DGAT1 compounds. These definitions apply to the terms as they are used throughout the specification unless they are otherwise limited in specific instances either individually or as part of a larger group, e.g., wherein an attachment point of a certain group is limited to a specific atom within that group.

The term “substituted or unsubstituted alkyl” refers to straight- or branched-chain hydrocarbon groups having 1-20 carbon atoms, preferably 1-10 carbon atoms, containing 0 to 3 substituents. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halo, hydroxy, alkanoyl, alkoxy, alkoxycarbonyl, alkoxycarbonyloxy, alkanoyloxy, thiol, alkylthio, alkylthiono, alkylsulfonyl, sulfamoyl, sulfonamido, carbamoyl, cyano, carboxy, acyl, aryl, alkenyl, alkynyl, aralkyl, aralkanoyl, aralkylthio, arylsulfonyl, arylthio, aroyl, aroyloxy, aryloxycarbonyl, aralkoxy, guanidino, optionally substituted amino, heterocyclyl.

The term “lower alkyl” refers to those alkyl groups as described above having 1-7, preferably 2-4 carbon atoms.

The term “halogen” or “halo” refers to fluorine, chlorine, bromine and iodine.

The term “alkenyl” refers to any of the above alkyl groups having at least two carbon atoms and further containing a carbon to carbon double bond at the point of attachment. Groups having 2-4 carbon atoms are preferred.

The term “alkynyl” refers to any of the above alkyl groups having at least two carbon atoms and further containing a carbon to carbon triple bond at the point of attachment. Groups having 2-4 carbon atoms are preferred.

The term “alkylene” refers to a straight-chain bridge of 4-6 carbon atoms connected by single bonds, e.g., —(CH₂)ₓ—, wherein x is 4-6, which may be interrupted with one or more heteroatoms selected from O, S, S(O), S(O)₂ or NR, wherein R may be hydrogen, alkyl, cycloalkyl, aryl, heterocyclyl, aralkyl, heteroaralkyl, acyl, carbamoyl, sulfonyl, alkoxycarbonyl, aryloxycarbonyl or aralkoxycarbonyl and the like; and the alkylene may further be substituted with one or more substituents selected from optionally substituted alkyl, cycloalkyl, aryl, heterocyclyl, oxo, halogen, hydroxy, carboxy, alkoxy, alkoxycarbonyl and the like.

The term “cycloalkyl” refers to optionally substituted monocyclic, bicyclic or tricyclic hydrocarbon groups of 3-12 carbon atoms, each of which may contain one or more carbon to carbon double bonds, or the cycloalkyl may be substituted by one or more substituents, such as alkyl, halo, oxo, hydroxy, alkoxy, alkanoyl, acylamino, carbamoyl, alkylamino, dialkylamino, thiol, alkylthio, cyano, carboxy, alkoxycarbonyl, sulfonyl, sulfonamido, sulfamoyl, heterocyclyl and the like.

The term “carboxamide” refers to —C(O)—NHR_{□}, wherein R_{□} is selected from hydrogen, a C₁-C₈ alkyl group, a cycloalkyl group, a substituted or unsubstituted aryl group, a substituted or unsubstituted heterocyclyl group, and carboxamide is preferably —C(O)—NH₂.

Exemplary monocyclic hydrocarbon groups include, but are not limited to, cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

The term “alkoxy” refers to alkyl-O—.

The term “alkanoyl” refers to alkyl-C(O)—.

The term “alkanoyloxy” refers to alkyl-C(O)—O—. The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “sulfamoyl” refers to H₂NS(O)₂—, alkyl-NHS(O)₂—, (alkyl)₂NS(O)₂—, aryl-NHS(O)₂, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

The term “sulfonamido” refers to alkyl-S(O)₂—NH—, aryl-S(O)₂—NH—, aralkyl-S(O)₂—NH—, heteroaryl-S(O)₂—NH—, heteroaralkyl-S(O)₂—NH—, alkyl-S(O)₂—N(alkyl)-, aryl-S(O)₂—N(alkyl)-, aralkyl-S(O)₂—N(alkyl)-, heteroaryl-S(O)₂—N(alkyl)-, heteroaralkyl-S(O)₂—N(alkyl)- and the like.

The term “sulfonyl” refers to alkylsulfonyl, arylsulfonyl, heteroarylsulfonyl, aralkylsulfonyl, heteroaralkylsulfonyl and the like.

The term “optionally substituted amino” refers to a primary or secondary amino group which may optionally be substituted by a substituent such as acyl, sulfonyl, alkoxycarbonyl, cycloalkoxycarbonyl, aryloxycarbonyl, heteroaryloxycarbonyl, aralkoxycarbonyl, heteroaralkoxycarbonyl, carbamoyl and the like.

The term “aryl” refers to monocyclic or bicyclic aromatic hydrocarbon groups having 6-12 carbon atoms in the ring portion, such as phenyl, biphenyl, naphthyl and tetrahydronaphthyl, each of which may optionally be substituted by 1-4 substituents, such as optionally substituted alkyl, trifluoromethyl, cycloalkyl, halo, hydroxy, alkoxy, acyl, alkanoyloxy, aryloxy, optionally substituted amino, thiol, alkylthio, arylthio, nitro, cyano, carboxy, alkoxycarbonyl, carbamoyl, alkylthiono, sulfonyl, sulfonamido, heterocyclyl and the like.

The term “monocyclic aryl” refers to optionally substituted phenyl as described under aryl.

The term “aralkyl” refers to an aryl group bonded directly through an alkyl group, such as benzyl.

The term “aralkanoyl” refers to aralkyl-C(O)—.

The term “aralkylthio” refers to aralkyl-S—.

The term “aralkoxy” refers to an aryl group bonded directly through an alkoxy group.

The term “arylsulfonyl” refers to aryl-S(O)₂—.

The term “arylthio” refers to aryl-S—.

The term “aroyl” refers to aryl-C(O)—.

The term “aroyloxy” refers to aryl-C(O)—O—.

The term “aroylamino” refers to aryl-C(O)—NH—.

The term “aryloxycarbonyl” refers to aryl-O—C(O)—.

The term “heterocyclyl” or “heterocyclo” refers to an optionally substituted, fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyridyl N-oxide, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” includes substituted heterocyclic groups. Substituted heterocyclic groups refer to heterocyclic groups substituted with 1, 2 or 3 substituents. Exemplary substituents include, but are not limited to, the following:
(a) optionally substituted alkyl;
(b) hydroxyl (or protected hydroxyl);
(c) halo;
(d) oxo, i.e., ═O;
(e) optionally substituted amino;
(f) alkoxy;
(g) cycloalkyl;
(h) carboxy;
(i) heterocyclooxy;
(j) alkoxycarbonyl, such as unsubstituted lower alkoxycarbonyl;
(k) mercapto;
(l) nitro;
(m) cyano;
(n) sulfamoyl;
(o) alkanoyloxy;
(p) aroyloxy;
(q) arylthio;
(r) aryloxy;
(s) alkylthio;
(t) formyl;
(u) carbamoyl;
(v) aralkyl; or
(w) aryl optionally substituted with alkyl, cycloalkyl, alkoxy, hydroxyl, amino, acylamino, alkylamino, dialkylamino or halo.

The term “heterocyclooxy” denotes a heterocyclic group bonded through an oxygen bridge.

The terms “saturated or unsaturated heterocycloalkyl” or “heterocycloalkyl” refers to nonaromatic heterocyclic or heterocyclyl groups as described above.

The term “heteroaryl” refers to an aromatic heterocycle, e.g., monocyclic or bicyclic aryl, such as pyrrolyl, pyrazolyl, imidazolyl, triazolyl, oxazolyl, isoxazolyl, thiazolyl, isothiazolyl, furyl, thienyl, pyridyl, pyridyl N-oxide, pyrazinyl, pyrimidinyl, pyridazinyl, indolyl, benzothiazolyl, benzoxazolyl, benzothienyl, quinolinyl, isoquinolinyl, benzimidazolyl, benzofuryl and the like, optionally substituted by, e.g., lower alkyl, lower alkoxy or halo.

The term “heteroarylsulfonyl” refers to heteroaryl-S(O)₂—.

The term “heteroaroyl” refers to heteroaryl-C(O)—.

The term “heteroaroylamino” refers to heteroaryl-C(O)NH—.

The term “heteroaralkyl” refers to a heteroaryl group bonded through an alkyl group.

The term “heteroaralkanoyl” refers to heteroaralkyl-C(O)—.

The term “heteroaralkanoylamino” refers to heteroaralkyl-C(O)NH—.

The term “acyl” refers to alkanoyl, aroyl, heteroaroyl, aralkanoyl, heteroaralkanoyl and the like.

The term “acylamino” refers to alkanoylamino, aroylamino, heteroaroylamino, aralkanoylamino, heteroaralkanoylamino and the like.

The term “divalent” refers to a residue linked to at least two residues and optionally having further substituents. As an example, within the context of the present invention the expression “substituted or unsubstituted divalent phenyl residue” is considered to be equivalent to the expression “substituted or unsubstituted phenylene residue”.

For example, a DGAT1 inhibitor compound having the following structure

A-L1-B-C-D-L2-E
and pharmaceutically acceptable salts, and prodrugs thereof, wherein
A is a substituted or unsubstituted alkyl, cycloalkyl, aryl, or heterocyclyl group,
L1 is selected from the group consisting of:
   an amine group —NH—,
   a substituted amine group of the formula —N(CH₃)—, —(CH₂—NH— or —CH₂—(CH₂—NH—,
   an amide group —C(O)—NH—,
   a sulphonamide group —S(O)₂—NH—, or
   a urea group —NHC(O)—NH—,
B is a substituted or unsubstituted, monocyclic, 5- or 6-membered divalent heteroaryl group,
C-D is selected from the following cyclic structures:
   C-D together is a substituted or unsubstituted divalent biphenyl group,
   C is a substituted or unsubstituted divalent phenyl group and D is a single bond,
   C is a substituted or unsubstituted divalent phenyl group, and D is a substituted or unsubstituted divalent non-aromatic monocyclic ring which is selected from a saturated or unsaturated divalent cycloalkyl group or a saturated or unsaturated divalent heterocycloalkyl group,
   C-D together is a spiro residue, wherein
      the first cyclic component is a benzo-fused cyclic component wherein the ring which is fused to the phenyl part is a 5- or 6-membered ring, optionally comprising one or more heteroatoms, the first cyclic component being attached to the moiety B via its phenyl part, and
      the second cyclic component is a cycloalkyl or cycloalkylidenyl residue which is attached to L2,
L2 is selected from the group consisting of:
   a single bond,
   a divalent residue having the following structure:

—[R¹]ₐ-[R²]_{b}—[C(O)]_{c}—[N(R³)]_{d}—[R⁴]_{c}—[R⁵]_{f}—
wherein
a is 0 or 1,
b is 0 or 1,
c is 0 or 1,
d is 0 or 1,
e is 0 or 1,
f is 0 or 1,
with the proviso that (a+b+c+d+e+f)>0, and c=1 if d=1, R¹, R², R⁴ and R⁵, which can be the same or different, are a substituted or unsubstituted divalent alkyl, cycloalkyl, alkenyl, alkynyl, alkylene, aryl or heterocyclyl residue,
R³ is H or hydrocarbyl,
or R³ and R⁴ form together with the nitrogen atom to which they are attached a 5- or 6-membered heterocycloalkyl group, with the proviso that R¹ and R² are not both alkyl if c=1 and d=e=f=0 and the carbonyl carbon atom is attached to the moiety E,
an alkylidenyl group which is linked to the moiety D via a double bond, and
E is selected from the group consisting of:
   a sulphonic acid group and derivatives thereof,
   a carboxyl group and derivatives thereof, wherein the carboxyl carbon atom is attached to L2,
   a phosphonic acid group and derivatives thereof,
   an alpha-keto hydroxyalkyl group,
   a hydroxyalkyl group wherein the carbon atom bonded to the hydroxyl group is further substituted with one or two trifluoro-methyl groups,
   a substituted or unsubstituted five-membered heterocyclyl residue having in the ring at least two heteroatoms and at least one carbon atom, wherein
      the at least one carbon atom of the ring is bonded to two heteroatoms;
      at least one of the heteroatoms to which the carbon atom of the ring is bonded is a member of the ring;
      and at least one of the heteroatoms to which the carbon atom of the ring is bonded or at least one of the heteroatoms of the ring is bearing a hydrogen atom;
with the provisos that
L2 is not a single bond or a divalent alkyl group if the moiety D is a single bond,
L2 is not a single bond if the moiety D is an unsubstituted divalent phenyl group and E is a carboxylic acid or a derivative thereof,
E is not a carboxamide group if L2 comprises an amide group,
E is not a —COOH group if D is a single bond and L2 is a —N(CH₃)—C(O)— group wherein the carbonyl carbon atom is attached to the moiety E,
L2 is not a divalent N-methyl piperidinyl group if the moiety E is a pyridinyl-1,2,4-triazolyl group,
L2 is not —C(O)-[R⁴]ₑ-[R⁵]_{f} when C is a substituted or unsubstituted divalent phenyl group and D is a single bond,
or a pharmaceutically acceptable salt thereof,

Compounds of the present invention may be prepared from commercially available reagents employing general synthetic techniques known to those skilled in the art.

WO 2007/126957 discloses the synthetic reaction schemes suitable for preparing such compounds. WO 2007/2007/126957 specifically disclose the following compounds.
(4-{4-[2-(3-Fluorophenylamino)-pyrimidin-5-yl]-phenyl}-cyclohexyl)-acetic acid,
{4-[4-(2-Phenylaminopyrimidin-5-yl)-phenyl]-cyclohexyl}-acetic acid,
4-{4-[2-(3-Fluorophenylamino)-pyrimidin-5-yl]-phenyl}-2,2-dimethyl-4-oxo-butyric acid,
(1S,2S)-2-{4-[2-(3-Fluorophenylamino)-pyrimidin-5-yl]-benzoyl}-cyclopentanecarboxylic acid,
(1S,2S)-2-{4-[2-(3-Chlorophenylamino)-pyrimidin-5-yl]-benzoyl}-cyclopentanecarboxylic acid,
(4-{4-[2-(3-Methoxyphenylamino)-thiazol-4-yl]-phenyl}-cyclohexyl)-acetic acid,
(4-{4-[2-(3-Fluorophenylamino)-thiazol-4-yl]-phenyl}-cyclohexyl)-acetic acid,
(4-{4-[2-(2-Chlorophenylamino)-thiazol-4-yl]-phenyl}-cyclohexyl)-acetic acid,
(4-{4-[2-(3-Cyanophenylamino)-thiazol-4-yl]-phenyl}-cyclohexyl)-acetic acid,
(4-{4-[2-(3-Trifluoromethylphenylamino)-thiazol-4-yl]-phenyl}-cyclohexyl)-acetic acid,
(4-{4-[2-(3-Fluorophenylamino)-thiazol-4-yl]-phenyl}-cyclohexyl)-acetic acid,
3-{4′-[2-(3-Fluorophenylamino)-thiazol-4-yl]-biphenyl-4-yl}-propionic acid,
{4′-[2-(3-Fluorophenylamino)-thiazol-4-yl]-biphenyl-4-yl}-acetic acid,
(4-{4-[2-(3-Chlorophenylamino)-oxazol-5-yl]-phenyl}-cyclohexyl)-acetic acid,
(4-{4-[2-(4-Chlorophenylamino)-oxazol-5-yl]-phenyl}-cyclohexyl)-acetic acid,
(4-{4-[2-(4-Methoxyphenylamino)-oxazol-5-yl]-phenyl}-cyclohexyl)-acetic acid,
(4-{4-[2-(2-Fluorophenylamino)-oxazol-5-yl]-phenyl}-cyclohexyl)-acetic acid,
{4-[4-(2-Phenylaminooxazol-5-yl)-phenyl]-cyclohexyl}-acetic acid,
(4-{4-[2-(3-Fluorophenylamino)-oxazol-5-yl]-phenyl}-cyclohexyl)-acetic acid,
(4-{4-[2-(2-Chlorophenylamino)-oxazol-5-yl]-phenyl}-cyclohexyl)-acetic acid,
(4-{4-[2-(3-Cyanophenylamino)-oxazol-5-yl]-phenyl}-cyclohexyl)-acetic acid,
{4-[4-(2-Cyclohexylaminooxazol-5-yl)-phenyl]-cyclohexyl}-acetic acid,
(4-{4-[2-(3,4-Dichlorophenylamino)-oxazol-5-yl]-phenyl}-cyclohexyl)-acetic acid,
(4-{4-[2-(3-Chloro-4-fluorophenylamino)-oxazol-5-yl]-phenyl}cyclohexyl)-acetic acid,
(4-{4-[2-(4-Chloro-3-trifluoromethylphenylamino)-oxazol-5-yl]-phenyl}-cyclohexyl)-acetic acid,
(4-{4-[2-(3,5-Difluorophenylamino)-oxazol-5-yl]-phenyl}-cyclohexyl)-acetic acid,
(4-{4-[2-(3,5-Dichlorophenylamino)-oxazol-5-yl]-phenyl}-cyclohexyl)-acetic acid,
(4-[4-{2-(2-Chloro-4-trifluoromethylphenylamino)-oxazol-5-yl]-phenyl}-cyclohexyl)-acetic acid,
(4-{4-[2-(2-Trifluoromethylphenylamino)-oxazol-5-yl]-phenyl}-cyclohexyl)-acetic acid,
(4-{4-[2-(3-Fluoro-4-methylphenylamino)-oxazol-5-yl]-phenyl}-cyclohexyl)-acetic acid,
{4-[4-(2-p-Tolylaminooxazol-5-yl)-phenyl]-cyclohexyl}-acetic acid,
(4-{4-[2-(3-Chloro-4-methylphenylamino)-oxazol-5-yl]-phenyl}-cyclohexyl)-acetic acid,
4-(4-{4-[2-(3-Chlorophenylamino)-oxazol-5-yl]-phenyl}-cyclohexyl)-butyric acid,
(E)-4-(4-{4-[2-(3-Chlorophenylamino)-oxazol-5-yl]-phenyl}-cyclohexyl)-but-2-enoic acid,
3-[2-(4-{4-[2-(3-Chlorophenylamino)-oxazol-5-yl]-phenyl}-cyclohexyl)-acetylamino]-propionic acid,
{[2-(4-{4-[2-(3-Chlorophenylamino)-oxazol-5-yl]-phenyl}-cyclohexyl)-acetyl]-methyl-amino}-acetic acid,
{4′-[2-(3-Chlorophenylamino)-oxazol-5-yl]-biphenyl-4-yl}-acetic acid,
3-{4′-[2-(3-Chlorophenylamino)-oxazol-5-yl]-biphenyl-4-yl}-propionic acid,
4-{4′-[2-(3-Chlorophenylamino)-oxazol-5-yl]-biphenyl-4-yl}-2,2-dimethyl-4-oxo-butyric acid,
4-{4′-[2-(3-Chlorophenylamino)-oxazol-5-yl]-biphenyl-4-yl}-4-oxo-butyric acid,
4-{4′-[2-(3-Chlorophenylamino)-oxazol-5-yl]-biphenyl-4-carbonyl}-cyclohexanecarboxylic acid,
(4-{4-[2-(3-Chlorophenylamino)-oxazol-5-yl]-phenyl}-3,6-dihydro-2H-pyridin-1-yl)-oxo-acetic acid,
4-{4-[2-(3-Chloro-phenylamino)-oxazol-5-yl]-phenyl}-3,6-dihydro-2H-pyridine-1-sulfonic acid amide,
4-{4-[2-(3-Chloro-phenylamino)-oxazol-5-yl]-phenyl}-3,6-dihydro-2H-pyridine-1-sulfonic acid amide-N-carboxylic acid tert-butyl ester,
4-(4-{4-[2-(3-Chloro-phenylamino)-oxazol-5-yl]-phenyl}-3,6-dihydro-2H-pyridin-1-yl)-2,2-dimethyl-4-oxo-butyric acid,
4-(4-{4-[2-(3-Chloro-phenylamino)-oxazol-5-yl]-phenyl}-3,6-dihydro-2H-pyridin-1-yl)-4-oxo-butyric acid,
2-(4-{4-[2-(3-Chloro-phenylamino-oxazol-5-yl]-phenyl}-3,6-dihydro-2H-pyridine-1-carbonyl)-benzoic acid,
(1R,2R)-2-{4′-[2-(3-Chlorophenylamino)-oxazol-5-yl]-biphenyl-4-carbonyl}-cyclohexanecarboxylic acid,
(trans)-2-{4′-[2-(3-Chlorophenylamino)-oxazol-5-yl]-biphenyl-4-carbonyl}-cyclohexanecarboxylic acid,
(trans)-2-{4′-[2-(3-Chlorophenylamino)-oxazol-5-yl]-biphenyl-4-carbonyl}-cyclopentanecarboxylic acid,
(4-{4′-[2-(3-Chloro-phenylamino)-oxazol-5-yl]-biphenyl-4-yl}-cyclohexyl)-acetic acid,
(4-{5-[6-(6-Trifluoromethyl-pyridin-3-ylamino)-pyridin-3-yl]-spirocyclohexylidenyl-1,1′-indanyl}-acetic acid,
(4-{5-[6-(6-Trifluoromethyl-pyridin-3-ylamino)-pyridin-3-yl]-spirocyclohexyl-1,1′-indanyl}-acetic acid,
(4-{4-[6-(3-Chloro-phenylamino)-pyridin-3-yl]-phenyl}-cyclohexyl)-acetic acid,
(4-{4-[6-(3-methylphenylamino)-pyridin-3-yl]-phenyl}-cyclohexyl)-acetic acid,
(4-{4-[6-(3-Trifluoromethylphenylamino)-pyridin-3-yl]-phenyl}-cyclohexyl)-acetic acid,
(4-{4-[6-(3-Methoxyphenylamino)-pyridin-3-yl]-phenyl}-cyclohexyl)-acetic acid,
(4-{4-[6-(2-Fluorophenylamino)-pyridin-3-yl]-phenyl}-cyclohexyl)-acetic acid,
(4-{4-[6-(2-Methoxyphenylamino)-pyridin-3-yl]-phenyl}-cyclohexyl)-acetic acid,
(4-{4-[6-(2-Methoxyphenylamino)-pyridin-3-yl]-phenyl}-cyclohexyl)-acetic acid,
(4-{4-[5-(6-Trifluoromethyl-pyridin-3-ylamino)-pyridin-2-yl]-phenyl}-cyclohexyl)-acetic acid,
(4-{4-[5-(Pyridin-2-ylamino)-pyridin-2-yl]-phenyl}-cyclohexyl)-acetic acid,
{4-[4-(5-Phenylaminopyridin-2-yl)-phenyl]-cyclohexyl}-acetic acid,
(4-{4-[5-(5-Cyanopyridin-3-ylamino)-pyridin-2-yl]-phenyl}-cyclohexyl)-acetic acid,
(4-{4-[5-(5-Trifluoromethylpyridin-2-ylamino)-pyridin-2-yl]-phenyl}-cyclohexyl)-acetic acid,
(4-{4-[5-(4-Trifluoromethylphenylamino)-pyridin-2-yl]-phenyl}-cyclohexyl)-acetic acid,
(4-{4-[5-(5-Methylpyridin-2-ylamino)-pyridin-2-yl]-phenyl}-cyclohexyl)-acetic acid,
(4-{4-[5-(5-Trifluoromethylpyridin-2-ylamino)-pyridin-2-yl]-phenyl}-cyclohexyl)-acetic acid methyl ester,
(4-{4-[5-(5-Chloropyridin-2-ylamino)-pyridin-2-yl]-phenyl}-cyclohexyl)-acetic acid,
(4-{4-[5-(6-Methoxypyridin-3-ylamino)-pyridin-2-yl]-phenyl}-cyclohexyl)-acetic acid,
(4-{4-[5-(5-Fluoropyridin-2-ylamino)-pyridin-2-yl]-phenyl}-cyclohexyl)-acetic acid,
(4-{4-[5-(6-Acetylaminopyridin-3-ylamino)-pyridin-2-yl]-phenyl}-cyclohexyl)-acetic acid,
{4-[4-(3-Methoxy-5-phenylamino-pyridin-2-yl)-phenyl]-cyclohexyl}-acetic acid,
{4-[4-(3-Methoxy-5-(3-fluorophenyl)amino-pyridin-2-yl)-phenyl]-cyclohexyl}-acetic acid,
{4-[4-(3-Methoxy-5-(4-trifluoromethyl-phenyl)amino-pyridin-2-yl)-phenyl]-cyclohexyl}-acetic acid,
{4-[4-(3-Methoxy-5-(3-chlorophenyl)amino-pyridin-2-yl)-phenyl]-cyclohexyl}-acetic acid,
(4-{4-[5-(3-Fluoro-phenylamino)-pyridin-2-yl]-phenyl}-cyclohexyl)-acetic acid,
(4-{4-[5-(3-Chloro-phenylamino)-pyridin-2-yl]-phenyl}-cyclohexyl)-acetic acid,
(4-{4-[5-(1-Methyl-1H-pyrazol-3-ylamino)-pyridin-2-yl]-phenyl}-cyclohexyl)-acetic acid,
(4-{4-[5-(5-Fluoro-6-methoxy-pyridin-3-ylamino)-pyridin-2-yl]-phenyl}-cyclohexyl)-acetic acid,
(4-{4-[5-(Isoxazol-3-ylamino)-pyridin-2-yl]-phenyl}-cyclohexyl)-acetic acid,
(4-{5-[5-(6-Trifluoromethyl-pyridin-3-ylamino)-pyridin-2-yl]-spirocyclohexylidenyl-1,1′-indanyl}-acetic acid,
(4-{4-[6-(3-Chloro-phenylamino)-pyridazin-3-yl]-phenyl}-cyclohexyl)-acetic acid,
(4-{4-[6-(3-Fluoro-phenylamino)-pyridazin-3-yl]-phenyl}-cyclohexyl)-acetic acid,
{4-[4-(6-m-Tolylamino-pyridazin-3-yl)-phenyl]-cyclohexyl}-acetic acid,
(4-{4-[6-(3-Trifluoromethyl-phenylamino)-pyridazin-3-yl]-phenyl}-cyclohexyl)-acetic acid,
(4-{4-[6-(3-Methoxy-phenylamino)-pyridazin-3-yl]-phenyl}-cyclohexyl)-acetic acid,
(4-{4-[6-(3-Cyano-phenylamino)-pyridazin-3-yl]-phenyl}-cyclohexyl)-acetic acid,
(4-{4-[6-(2-Fluoro-phenylamino)-pyridazin-3-yl]-phenyl}-cyclohexyl)-acetic acid,
(4-{4-[6-(4-Chloro-phenylamino)-pyridazin-3-yl]-phenyl}-cyclohexyl)-acetic acid,
{4-[4-(6-p-Tolylamino-pyridazin-3-yl)-phenyl]-cyclohexyl}-acetic acid,
(4-{4-[6-(4-Trifluoromethyl-phenylamino)-pyridazin-3-yl]-phenyl}-cyclohexyl)-acetic acid,
(4-{4-[6-(3-Chloro-4-methoxy-phenylamino)-pyridazin-3-yl]-phenyl}-cyclohexyl)-acetic acid,
(4-{4-[6-(3-Chloro-2-methyl-phenylamino)-pyridazin-3-yl]-phenyl}-cyclohexyl)-acetic acid,
{4-[4-(6-Phenylamino-pyridazin-3-yl)-phenyl]-cyclohexyl}-acetic acid,
(4-{4-[6-(3-Chloro-2-methoxy-phenylamino)-pyridazin-3-yl]-phenyl}-cyclohexyl)-acetic acid,
(4-{4-[6-(2-Methoxy-phenylamino)-pyridazin-3-yl]-phenyl}-cyclohexyl)-acetic acid,
(4-{4-[6-(4-Methoxy-phenylamino)-pyridazin-3-yl]-phenyl}-cyclohexyl)-acetic acid,
(4-{4-[6-(6-Trifluoromethyl-pyridin-3-ylamino)-pyridazin-3-yl]-phenyl}-cyclohexyl)-acetic acid,
(4-{4-[6-(4-Trifluoromethoxy-phenylamino)-pyridazin-3-yl]-phenyl}-cyclohexyl)-acetic acid,
(4-{4-[6-(4-Fluoro-phenylamino)-pyridazin-3-yl]-phenyl}-cyclohexyl)-acetic acid,
(4-{4-[6-(6-Amino-pyridin-3-ylamino)-pyridazin-3-yl]-phenyl}-cyclohexyl)-acetic acid,
(4-{4-[6-(Methyl-m-tolyl-amino)-pyridazin-3-yl]-phenyl}-cyclohexyl)-acetic acid,
[4-(4-{6-[(3-Chloro-phenyl)-methyl-amino]-pyridazin-3-yl}-phenyl)-cyclohexyl]-acetic acid,
[4-(4-{6-[(3-Methoxy-phenyl)-methyl-amino]-pyridazin-3-yl}-phenyl)-cyclohexyl]-acetic acid,
(4-{4-[6-(2-Methyl-6-trifluoromethyl-pyridin-3-ylamino)-pyridazin-3-yl]-phenyl}-cyclohexyl)-acetic acid,
(4-{4-[6-(3-Chloro-2-methoxy-phenylamino)-pyridazin-3-yl]-phenyl}-cyclohexyl)-acetic acid,
2-{4-[6-(3-Chloro-phenylamino)-pyridazin-3-yl]-benzoylamino}-3-methyl-butyric acid,
(S)-1-{4-[6-(3-Chloro-phenylamino)-pyridazin-3-yl]-benzoyl}-pyrrolidine-2-carboxylic acid,
(1S,2R)-2-{4-[6-(3-Chloro-phenylamino)-pyridazin-3-yl]-benzoylamino}-cyclopentanecarboxylic acid,
3-{4-[6-(3-Chloro-phenylamino)-pyridazin-3-yl]-benzoylamino}-propionic acid,
(S)-3-{4-[6-(3-Chloro-phenylamino)-pyridazin-3-yl]-benzoylamino}-5-methyl-hexanoic acid,
(1S,2R)-2-{4-[6-(3-Chloro-phenylamino)-pyridazin-3-yl]-benzoylamino}-cyclohexanecarboxylic acid,
(S)-1-{4-[6-(3-Chloro-phenylamino)-pyridazin-3-yl]-benzoyl}-piperidine-2-carboxylic acid,
2-{4-[6-(3-Chloro-phenylamino)-pyridazin-3-yl]-benzoylamino}-2-methyl-propionic acid,
4-{4-[6-(3-Trifluoromethyl-phenylamino)-pyridazin-3-yl]-phenyl}-cyclohexanecarboxylic acid,
2-(4-{4-[6-(3-Chloro-phenylamino)-pyridazin-3-yl]-phenyl}-cyclohexyl)-acetamide,
(6-{4-[4-(2H-Tetrazol-5-ylmethyl)-cyclohexyl]-phenyl}-pyridazin-3-yl)-(6-trifluoromethyl-pyridin-3-yl)-amine,
3-(4-{4-[6-(6-Trifluoromethyl-pyridin-3-ylamino)-pyridazin-3-yl]-phenyl}-cyclohexylmethyl)-4H-[1,2,4]oxadiazol-5-one,
(1-{4-[6-(3-Trifluoromethyl-phenylamino)-pyridazin-3-yl]-phenyl}-piperidin-4-yl)-acetic acid,
(4-{4-[4-Methyl-6-(6-trifluoromethyl-pyridin-3-ylamino)-pyridazin-3-yl]-phenyl}-cyclohexyl)-acetic acid,
(4-{4-[4-Methyl-6-(4-trifluoromethyl-phenylamino)-pyridazin-3-yl]-phenyl}-cyclohexyl)-acetic acid
(4-{4-[5-(6-Trifluoromethyl-pyridin-3-ylamino)-pyrazin-2-yl]-phenyl}-cyclohexyl)-acetic acid,
(4-{4-[5-(2,2-Dimethyl-propionylamino)-pyridin-2-yl]-phenyl}-cyclohexyl)-acetic acid,
(4-{4-[5-(Benzooxazol-2-ylamino)-pyridin-2-yl]-phenyl}-cyclohexyl)-acetic acid,
(4-{4-[6-(6-Methoxy-pyridin-3-ylamino)-5-methyl-pyridin-3-yl]-phenyl}-cyclohexyl)-acetic acid,
(4-{4-[5-Fluoro-6-(6-methoxy-pyridin-3-ylamino)-pyridin-3-yl]-phenyl}-cyclohexyl)-acetic acid,
Oxo-(4-{4-[6-(6-trifluoromethyl-pyridin-3-ylamino)-pyridin-3-yl]-phenyl}-cyclohexyl)-acetic acid,
{4-[4-(5-Acetylamino-pyridin-2-yl)-phenyl]-cyclohexyl}-acetic acid,
(4-{4-[5-(3-Trifluoromethyl-benzoylamino)-pyridin-2-yl]-phenyl}-cyclohexyl)-acetic acid,
[4-(4-{5-[(Pyridine-2-carbonyl)-amino]-pyridin-2-yl}-phenyl)-cyclohexyl]-acetic acid,
[4-(4-{5-[3-(4-Trifluoromethoxy-phenyl)-ureido]-pyridin-2-yl}-phenyl)-cyclohexyl]-acetic acid,
[4-(4-{5-[3-(2-Trifluoromethyl-phenyl)-ureido]-pyridin-2-yl}-phenyl)-cyclohexyl]-acetic acid,
(4-{4-[5-(3-o-Tolyl-ureido)-pyridin-2-yl]-phenyl}-cyclohexyl)-acetic acid,
[4-(4-{5-[(1-Methyl-1H-indole-3-carbonyl)-amino]-pyridin-2-yl}-phenyl)-cyclohexyl]-acetic acid,
[4-(4-{5-[(1H-Indole-3-carbonyl)-amino]-pyridin-2-yl}-phenyl)-cyclohexyl]-acetic acid,
[4-(4-{5-[(Pyridine-3-carbonyl)-amino]-pyridin-2-yl}-phenyl)-cyclohexyl]-acetic acid,
[4-(4-{5-[(6-Methyl-pyridine-3-carbonyl)-amino]-pyridin-2-yl}-phenyl)-cyclohexyl]-acetic acid,
[4-(4-{5-[(5-Bromo-pyridine-3-carbonyl)-amino]-pyridin-2-yl}-phenyl)-cyclohexyl]-acetic acid,
[4-(4-{5-[(5-Chloro-6-methoxy-pyridine-3-carbonyl)-amino]-pyridin-2-yl}-phenyl)-cyclohexyl]-acetic acid,
[4-(4-{5-[(5-Isobutyl-isoxazole-3-carbonyl)-amino]-pyridin-2-yl}-phenyl)-cyclohexyl]-acetic acid,
[4-(4-{5-[(3-tert-Butyl-1-methyl-1H-pyrazole-4-carbonyl)-amino]-pyridin-2-yl}-phenyl)-cyclohexyl]-acetic acid,
[4-(4-{5-[(5-tert-Butyl-1H-pyrazole-3-carbonyl)-amino]-pyridin-2-yl}-phenyl)-cyclohexyl]-acetic acid,
[4-(4-{5-[(5-Isopropyl-isoxazole-3-carbonyl)-amino]-pyridin-2-yl}-phenyl)-cyclohexyl]-acetic acid,
{4-[4-(5-Isobutoxycarbonylamino-pyridin-2-yl)-phenyl]-cyclohexyl}-acetic acid,
[4-(4-{5-[((S)-5-Oxo-pyrrolidine-2-carbonyl)-amino]-pyridin-2-yl}-phenyl)-cyclohexyl]-acetic acid,
(4-{4-[5-(4-Fluoro-3-trifluoromethyl-benzoylamino)-pyridin-2-yl]-phenyl}-cyclohexyl)-acetic acid,
(4-{4-[5-(4-Trifluoromethyl-benzoylamino)-pyridin-2-yl]-phenyl}-cyclohexyl)-acetic acid,
[4-(4-{5-[(6-Trifluoromethyl-pyridine-3-carbonyl)-amino]-pyridin-2-yl}-phenyl)-cyclohexyl]-acetic acid,
(4-{4-[5-(3-Fluoro-5-trifluoromethyl-benzoylamino)-pyridin-2-yl]-phenyl}-cyclohexyl)-acetic acid,
[4-(4-{5-[(Tetrahydro-pyran-4-carbonyl)-amino]-pyridin-2-yl}-phenyl)-cyclohexyl]-acetic acid,
[4-(4-{5-[(5-Bromo-2-methoxy-pyridine-3-carbonyl)-amino]-pyridin-2-yl}-phenyl)-cyclohexyl]-acetic acid,
[4-(4-{5-[(1,5-Dimethyl-1H-pyrazole-3-carbonyl)-amino]-pyridin-2-yl}-phenyl)-cyclohexyl]-acetic acid,
[4-(4-{5-[(5-Methoxy-1H-indole-3-carbonyl)-amino]-pyridin-2-yl}-phenyl)-cyclohexyl]-acetic acid,
[4-(4-{5-[(2,5-Dimethyl-1H-pyrrole-3-carbonyl)-amino]-pyridin-2-yl}-phenyl)-cyclohexyl]-acetic acid,
[4-(4-{5-[(1-Methyl-5-trifluoromethyl-1H-pyrazole-4-carbonyl)-amino]-pyridin-2-yl}-phenyl)-cyclohexyl]-acetic acid,
{4-[4-(5-{[4-(Morpholine-4-sulfonyl)-1H-pyrrole-2-carbonyl]-amino}-pyridin-2-yl)-phenyl]-cyclohexyl}-acetic acid,
(4-{4-[5-(2-Fluoro-2-methyl-propionylamino)-pyridin-2-yl]-phenyl}-cyclohexyl)-acetic acid,
[4-(4-{5-[(1-Methyl-3-trifluoromethyl-1H-pyrazole-4-carbonyl)-amino]-pyridin-2-yl}-phenyl)-cyclohexyl]-acetic acid methyl ester,
(4-{4-[5-(2-Methyl-2-pyrazol-1-yl-propionylamino)-pyridin-2-yl]-phenyl}-cyclohexyl)-acetic acid,
[4-(4-{5-[(5-Isopropyl-isoxazole-4-carbonyl)-amino]-pyridin-2-yl}-phenyl)-cyclohexyl]-acetic acid,
[4-(4-{5-[(1-Methyl-3-trifluoromethyl-1H-pyrazole-4-carbonyl)-amino]-pyridin-2-yl}-phenyl)-cyclohexyl]-acetic acid,
[4-(4-{5-[(5-Cyclopropyl-isoxazole-4-carbonyl)-amino]-pyridin-2-yl}-phenyl)-cyclohexyl]-acetic acid,
[4-(4-{5-[(5-Cyclopropyl-isoxazole-4-carbonyl)-amino]-pyridin-2-yl}-phenyl)-cyclohexyl]-acetic acid methyl ester,
[4-(4-{5-[(5-Cyclopropyl-isoxazole-3-carbonyl)-amino]-pyridin-2-yl}-phenyl)-cyclohexyl]-acetic acid,
[4-(4-{5-[(6-Methoxy-pyridine-3-carbonyl)-amino]-pyridin-2-yl}-phenyl)-cyclohexyl]-acetic acid,
(4-{4-[5-(2,2-Dimethyl-butyrylamino)-pyridin-2-yl]-phenyl}-cyclohexyl)-acetic acid,
(4-{4-[5-(2-Methoxy-2-methyl-propionylamino)-pyridin-2-yl]-phenyl}-cyclohexyl)-acetic acid,
[4-(4-{5-[(1,5-Dimethyl-1H-pyrazole-4-carbonyl)-amino]-pyridin-2-yl}-phenyl)-cyclohexyl]-acetic acid,
(4-{4-[5-(Tetrahydro-pyran-4-yloxycarbonylamino)-pyridin-2-yl]-phenyl}-cyclohexyl)-acetic acid,
{4-[4-(5-Cyclopropylmethoxycarbonylamino-pyridin-2-yl)-phenyl]-cyclohexyl}-acetic acid,
(4-{4-[5-(Tetrahydro-furan-2-ylmethoxycarbonylamino)-pyridin-2-yl]phenyl}-cyclohexyl)-acetic acid,
(4-{4-[5-(Tetrahydro-pyran-2-ylmethoxycarbonylamino)-pyridin-2-yl]-phenyl}-cyclohexyl)-acetic acid,
(4-{4-[5-(3-Methyl-oxetan-3-ylmethoxycarbonylamino)-pyridin-2-yl]-phenyl}-cyclohexyl)-acetic acid,
(4-{4-[5-(Tetrahydro-pyran-4-ylmethoxycarbonylamino)-pyridin-2-yl]-phenyl}cyclohexyl)-acetic acid,
(4-{4-[5-(2-Methyl-pyridin-3-ylmethoxycarbonylamino)-pyridin-2-yl]-phenyl}-cyclohexyl)-acetic acid,
[4-(4-{5-[3-(4-Chloro-3-trifluoromethyl-phenyl)-ureido]-pyridin 2-yl]-phenyl}-cyclohexyl)-acetic acid,
{4-[4-(5-Isopropylcarbamoyl-pyridin-2-yl)-phenyl]-cyclohexyl}-acetic acid,
{4-[4-(6-Carbamoyl-pyridin-2-yl)-phenyl]-cyclohexyl}-acetic acid,
{4-[4-(6-Isopropylcarbamoyl-pyridin-2-yl)-phenyl]-cyclohexyl}-acetic acid,
(4-{4-[5-(6-Trifluoromethyl-pyridin-3-ylcarbamoyl)-pyridin-2-yl]-phenyl}-cyclohexyl)-acetic acid,
(4-{4-[5-(4-Trifluoromethyl-benzenesulfonylamino)-pyridin-2-yl]-phenyl}-cyclohexyl)-acetic acid,
(4-{4-[5-(3-Trifluoromethyl-benzenesulfonylamino)-pyridin-2-yl]-phenyl}-cyclohexyl)-acetic acid,
(4-{4-[5-(1,2-Dimethyl-1H-imidazole-4-sulfonylamino)-pyridin-2-yl]-phenyl}-cyclohexyl)-acetic acid,
(4-{4-[5-(5-Fluoro-pyridin-3-ylamino)-pyridin-2-yl]-phenyl}-cyclohexyl)-acetic acid,
(4-{4-[5-(6-Isopropoxy-pyridin-3-ylamino)-pyridin-2-yl]-phenyl}-cyclohexyl)-acetic acid,
(4-{4-[5-(5-Bromo-pyridin-2-ylamino)-pyridin-2-yl]-phenyl}-cyclohexyl)-acetic acid,
(4-{4-[5-(2-Methoxy-pyrimidin-5-ylamino)-pyridin-2-yl]-phenyl}-cyclohexyl)-acetic acid,
(4-{4-[5-(6-Methylsulfanyl-pyridin-3-ylamino)-pyridin-2-yl]-phenyl}-cyclohexyl)-acetic acid,
(4-{4-[5-([1,2,4]Triazin-3-ylamino)-pyridin-2-yl]-phenyl}-cyclohexyl)-acetic acid,
(4-{4-[5-(2-Dimethylamino-pyrimidin-5-ylamino)-pyridin-2-yl]-phenyl}-cyclohexyl)-acetic acid,
(4-{4-[5-(5-Methylsulfanyl-pyridin-2-ylamino)-pyridin-2-yl]-phenyl}-cyclohexyl)-acetic acid,
(4-{4-[5-(3,5-Difluoro-pyridin-2-ylamino)-pyridin-2-yl]-phenyl}-cyclohexyl)-acetic acid,
(4-{4-[5-(6-Trifluoromethyl-pyridin-3-ylamino)-pyridin-2-yl]-phenyl}-cyclohexyl)-acetic acid methyl ester,
(4-{4-[5-(5-Chloro-6-methoxy-pyridin-3-ylamino)-pyridin-2-yl]-phenyl}-cyclohexyl)-acetic acid,
(4-{4-[(3-Fluoro-4-methyl-pyridin-2-ylamino)-pyridin-2-yl]-phenyl}-cyclohexyl)-acetic acid,
(4-{4-[5-(3-Chloro-1-methyl-pyridin-2-ylamino)-pyridin-2-yl]-phenyl}-cyclohexyl)-acetic acid,
(4-{4-[5-(5-Difluoromethyl-6-methoxy-pyridin-3-ylamino)-pyridin-2-yl]-phenyl}-cyclohexyl)-acetic acid,
(4-{4-[5-(5-Methanesulfonyl-pyridin-2-ylamino)-pyridin-2-yl]-phenyl}-cyclohexyl)-acetic acid,
(4-{4-[3-Fluoro-5-(6-trifluoromethyl-pyridin-3-ylamino)-pyridin-2-yl]-phenyl}-cyclohexyl)-acetic acid,
(4-{4-[5-(1H-Benzoimidazol-2-ylamino)-pyridin-2-yl]-phenyl}-cyclohexyl)-acetic acid,
(4-{4-[5-(5-Trifluoromethyl-[1,3,4]oxadiazol-2-ylamino)-pyridin-2-yl]-phenyl}-cyclohexyl)-acetic acid,
(4-{4-[5-(6-Methyl-benzooxazol-2-ylamino)-pyridin-2-yl]-phenyl}-cyclohexyl)-acetic acid,
(4-{4-[5-(2-Methyl-5-trifluoromethyl-2H-pyrazol-3-ylamino)-pyridin-2-yl]-phenyl}-cyclohexyl)-acetic acid,
(4-{4-[5-(6-Chloro-benzooxazol-2-ylamino)-pyridin-2-yl]-phenyl}-cyclohexyl)-acetic acid methyl ester,
(4-{4-[5-(6-Chloro-benzooxazol-2-ylamino)-pyridin-2-yl]-phenyl}-cyclohexyl)-acetic acid,
(4-{4-[5-(5-Chloro-6-methoxy-benzooxazol-2-ylamino)-pyridin-2-yl]-phenyl}-cyclohexyl)-acetic acid,
(4-{4-[5-(5-tert-Butyl-[1,3,4]oxadiazol-2-ylamino)-pyridin-2-yl]-phenyl}-cyclohexyl)-acetic acid,
(4-{4-[2-(6-Trifluoromethyl-pyridin-3-ylamino)-pyrimidin-5-yl]-phenyl}-cyclohexyl)-acetic acid,
(4-{4-[2-(5-Chloro-pyridin-2-ylamino)-pyrimidin-5-yl]-phenyl}-cyclohexyl)-acetic acid
Oxo-(4-{4-[6-(6-trifluoromethyl-pyridin-3-ylamino)-pyridin-3-yl]-phenyl}-piperidin-1-yl)-acetic acid,
(4-Hydroxy-4-{4-[6-(6-trifluoromethyl-pyridin-3-ylamino)-pyridin-3-yl]-phenyl}-piperidin-1-yl)-acetic acid,
(4-{4-[6-(2-Methyl-6-trifluoromethyl-pyridin-3-ylamino)-pyridin-3-yl]-phenyl}-cyclohexyl)-acetic acid,

or in any case a pharmaceutically acceptable salt thereof.

Another example of DGAT1 compounds are disclosed in International Patent Application PCT/US2007/081607. The disclosed compounds have the following basic formula.

A-L1-B-C-D
wherein
   A is selected from a substituted or unsubstituted alkyl, substituted or unsubstituted alkoxy, substituted or unsubstituted cycloalkyl, substituted or unsubstituted aryl, and a substituted or unsubstituted heterocyclyle, wherein A is linked to L1 via a carbon member of the ring when A is a ring,
   L1 is selected from the group consisting of:
      an amine group of the formula —(CH₂)ₙ—(CR₄R₄)ₚ—(CH₂)ₘ N(R₃)—,
      a thiocarbamoyl group of the formula —(CH₂)ₙ—(CR₄R_{4′})ₚ(CH₂)ₘ N(R₃)—C(S)—,
      an amide group of the formula —C(O)—N(R₃)—(CH₂)ₙ—(CR₄R_{4′})ₚ—(CH₂)ₘ—,
      an amidine group of the formula —C(NH)—N(R₃)—(CH₂)ₙ—(CR₄R₄)ₚ—(CH₂)ₘ₋₁,
      an amide group of the formula —(CH₂)ₙ—(CR₄R_{4′})ₚ—(CH₂)ₘ—C(O)—N(R₃)—,
      a sulphonamide group of the formula —(CH₂)ₙ (CR₄R_{4′})ₚ—(CH₂)ₘ —S(O)₂—N(R₃)—,
      a carbamate group of the formula —(CH₂)ₙ—(CR₄R_{4′})ₚ—(CH₂)ₘ—(O)—C(O)—N(R₃)—, or
      a urea group of the formula (CH₂)ₙ—(CR₄R₄)ₚ—(CH₂)ₘ—N(R₃)—C(O)—N(R_{3A})—,
wherein;
   R₃ and R_{3A} are, independently from each other, hydrogen or lower alkyl,
   m, n and p are, independently from each other, zero or an integer from 1 to 2,
   m+m+p is between 0 and 6, and is preferably 0, 1, 2 or 3
   R₄ and R_{4′} are, independently from each other, hydrogen, halogen, hydroxyl, lower alkoxy, lower alkoxycarbonyl, carboxy or lower alkyl, or R₄ and R_{4′} are joined together to form a Spiro residue of the formula

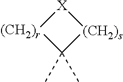

wherein;
   X is NR_{3′}, O, S or CR_{3″}R_{4″}
   r and s are, independently from each other, zero or an integer from 1 to 3,
   R₃ is hydrogen or lower alkyl,
   R_{3″} is hydrogen, halogen, hydroxyl, alkoxy, or lower alkyl,
   R_{4″} is hydrogen or lower alkyl;
B is a substituted or unsubstituted divalent heteroaryl group selected from one of the groups below:

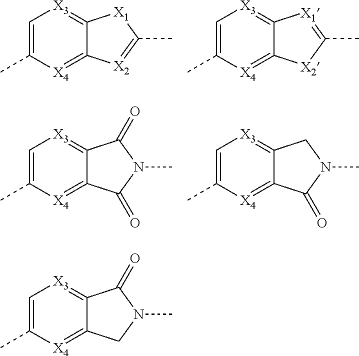

wherein;
X₁ and X₂′ are independently selected from O, NH, NR₉ or S, wherein R₉ is selected from lower alkyl, lower alkylamino, lower alkoxyalkyl, lower hydroxyalkyl,
X₁′, X₂, X₃ and X₄ are independently selected from N, or CH,
   C is

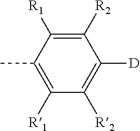

wherein
   —R₁ is selected from hydrogen, cyano, lower alkylsulfonylamino, alkanoylamino, halogen, lower alkyl, trifluoromethyl, lower alkoxy, lower alkylamino, lower dialkylamino, and NO₂,
   R′₁, R₂ and R′₂ are independently selected from hydrogen, halogen, trifluoromethyl, aryloxy, lower alkyl, lower alkoxy, lower alkylamino, lower dialkylamino, and NO₂,
or
   C may also be a substituted or unsubstituted bicyclic aryl or heteroaryl group,
   D is selected from hydrogen, halogen, hydroxyl, cyano, alkanoylamino, carboxy, carbamoyl, —O-L₂-E, —S— L₂-E′, —C(O)—O— L₂-E, -L₂-E″, and —NR₆-L₂-E′,
   L₂ is —(CH₂)_{n′}—(CR₅R_{5′})ₚ, —(CH₂)ₘ′
   E is;
      alkyl, acyl, alkoxycarbonyl, phosphonic acid, phosphonate, cycloalkoxycarbonyl, aryloxycarbonyl, heterocyclyloxycarbony, carboxy, carbamoyl, sulfonyl, —SO₂—OH, sulfamoyl, sulfonylcarbamoyl, sulfonyloxy, sulfonamido, —C(O)—O—R—PRO, substituted or unsubstituted aryl, substituted or unsubstituted heterocyclyl, or substituted or unsubstituted heteroaryl, and when n′+m′+p′ is equal to zero, E is not sulfonyloxy or sulfonamido,
   E′ is;
      alkyl, acyl, alkoxycarbonyl, cycloalkoxycarbonyl, aryloxycarbonyl, heterocyclyloxycarbony, carboxy, carbamoyl, sulfonylcarbamoyl, sulfonyl, —SO₂—OH, sulfamoyl, sulfonamido, phosphonic acid, phosphonate, sulfonyloxy, —C(O)—O—R—PRO, substituted or unsubstituted aryl, substituted or unsubstituted heterocyclyl, or substituted or unsubstituted heteroaryl, and when n′+m′+p′ is equal to zero, E′ is not sulfamoyl, sulfonamido, phosphonic acid, phosphonate, or sulfonyloxy,
   E″ is;
      alkyl, acyl, alkoxycarbonyl, phosphonic acid, phosphonate, cycloalkoxycarbonyl, aryloxycarbonyl, heterocyclyloxycarbony, carboxy, carbamoyl, sulfonyl, sulfamoyl, sulfonyloxy, sulfonamido, —SO₂—OH, sulfonylcarbamoyl, —C(O)—O—R—PRO, substituted or unsubstituted aryl, substituted or unsubstituted heterocyclyl, or substituted or unsubstituted heteroaryl,
   m′, n′ and p′ are, independently from each other, an integer from 0 to 4,
   m′+n′+p′ is between 0 and 12, and is preferably 0, 1, 2, 3 or 4,
   R₅ and R_{5′} are, independently from each other, hydrogen, halogen, hydroxyl, lower alkoxy, or lower alkyl, or R₅ and R_{5′} are joined together to form a spiro residue of the formula

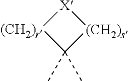

wherein;
   X′ is NRₓ, O, S or CR_{x′}R_{x″}
   r′ and s′ are, independently from each other, zero or an integer from 1 to 3,
   Rₓ is hydrogen or lower alkyl,
   Rₓ is hydrogen, halogen, hydroxyl, alkoxy, or lower alkyl,
   Rₓ is hydrogen or lower alkyl;
      or a prodrug or a pharmaceutically acceptable salt thereof for the manufacture of a medicament for the treatment of DGAT especially DGAT1 associated disorders.

Particular compounds of PCT/US2007/081607 are:
[2-(2-Chloro-phenyl)-3H-benzoimidazol-5-yl]-carbamic acid ethyl ester
[2-(4-Methoxy-2-methyl-phenyl)-3H-benzoimidazol-5-yl]-carbamic acid ethyl ester
[2-(2,6-Dimethyl-phenyl)-3H-benzoimidazol-5-yl]-carbamic acid ethyl ester
[2-(2,4-Dichloro-phenyl)-3H-benzoimidazol-5-yl]-carbamic acid ethyl ester
[2-(2,3-Dichloro-phenyl)-3H-benzoimidazol-5-yl]-carbamic acid ethyl ester
N-[2-(2,6-Dichloro-phenyl)-3H-benzoimidazol-5-yl]-butyramide
N-[2-(2,6-Dichloro-phenyl)-3H-benzoimidazol-5-yl]-3-methyl-butyramide
N-[2-(2,6-Dichloro-phenyl)-3H-benzoimidazol-5-yl]-2-ethoxy-acetamide
N-[2-(2,6-Dichloro-phenyl)-3H-benzoimidazol-5-yl]-3-methyl-benzamide
N-[2-(2,6-Dichloro-phenyl)-3H-benzoimidazol-5-yl]-2,4,6-trimethyl-benzenesulfonamide
2-(2,6-Dichloro-phenyl)-3H-benzoimidazole-5-carboxylic acid propylamide
2-(2,6-Dichloro-phenyl)-3H-benzoimidazole-5-carboxylic acid butylamide
2-(2,6-Dichloro-phenyl)-3H-benzoimidazole-5-carboxylic acid benzylamide
2-(2,6-Dichloro-phenyl)-3H-benzoimidazole-5-carboxylic acid (2-methoxy-ethyl)-amide
2-(2,6-Dichloro-phenyl)-3H-benzoimidazole-5-carboxylic acid isopropylamide
2-(2,6-Dichloro-phenyl)-3H-benzoimidazole-5-carboxylic acid cyclohexylamide
2-(2,6-Dichloro-phenyl)-3H-benzoimidazole-5-carboxylic acid isobutyl-methyl-amide
2-(2,6-Dichloro-phenyl)-3H-benzoimidazole-5-carboxylic acid diethylamide
2-(2,6-Dichloro-phenyl)-3H-benzoimidazole-5-carboxylic acid benzyl-methyl-amide
2-(2,6-Dichloro-phenyl)-3H-benzoimidazole-5-carboxylic acid ((R)-1-phenyl-ethyl)-amide
2-(2,6-Dichloro-phenyl)-3H-benzoimidazole-5-carboxylic acid ((S)-1-phenyl-ethyl)-amide
2-(2,6-Dichloro-phenyl)-3H-benzoimidazole-5-carboxylic acid (1,2,3,4-tetrahydro-naphthalen-1-yl)-amide
2-(2,6-Dichloro-phenyl)-3H-benzoimidazole-5-carboxylic acid (Rindan-1-ylamide
2-(2,6-Dichloro-phenyl)-3H-benzoimidazole-5-carboxylic acid (biphenyl-3-ylmethyl)-amide
2-(2,6-Dichloro-phenyl)-3H-benzoimidazole-5-carboxylic acid (biphenyl-4-ylmethyl)-amide
2-(2,6-Dichloro-phenyl)-3H-benzoimidazole-5-carboxylic acid 2-methyl-benzylamide
2-(2,6-Dichloro-phenyl)-3H-benzoimidazole-5-carboxylic acid phenethyl-amide
2-(2,6-Dichloro-phenyl)-3H-benzoimidazole-5-carboxylic acid (2-o-tolyl-ethyl)-amide
2-(2,6-Dichloro-phenyl)-3H-benzoimidazole-5-carboxylic acid phenylamide
2-(2,6-Dichloro-phenyl)-3H-benzoimidazole-5-carboxylic acid o-tolylamide
2-(2,6-Dichloro-phenyl)-3H-benzoimidazole-5-carboxylic acid (3-chloro-phenyl)-amide
2-(2,6-Dichloro-phenyl)-3H-benzoimidazole-5-carboxylic acid (4-chloro-phenyl)-amide
2-(2,6-Dichloro-phenyl)-3H-benzoimidazole-5-carboxylic acid (4-dimethylcarbamoyl-phenyl)-amide
2-(2,6-Dichloro-phenyl)-3H-benzoimidazole-5-carboxylic acid (3-methoxy-phenyl)-amide
2-(2,6-Dichloro-phenyl)-3H-benzoimidazole-5-carboxylic acid (4-methoxy-phenyl)-amide
2-(2,6-Dichloro-phenyl)-3H-benzoimidazole-5-carboxylic acid (3-isopropoxy-phenyl)-amide
2-(2,6-Dichloro-phenyl)-3H-benzoimidazole-5-carboxylic acid (3-ethoxy-phenyl)-amide
2-(2,6-Dichloro-phenyl)-3H-benzoimidazole-5-carboxylic acid (3,4-dimethyl-phenyl)-amide
2-(2,6-Dichloro-phenyl)-3H-benzoimidazole-5-carboxylic acid (3,5-dimethyl-phenyl)-amide
2-(2,6-Dichloro-phenyl)-3H-benzoimidazole-5-carboxylic acid p-tolylamide
2-(2,6-Dichloro-phenyl)-3H-benzoimidazole-5-carboxylic acid (3-cyano-phenyl)-amide
2-(2,6-Dichloro-phenyl)-3H-benzoimidazole-5-carboxylic acid (3-acetyl-phenyl)-amide
2-(2,6-Dichloro-phenyl)-3H-benzoimidazole-5-carboxylic acid (4-fluoro-phenyl)-amide
2-(2,6-Dichloro-phenyl)-3H-benzoimidazole-5-carboxylic acid (4-cyano-phenyl)-amide
2-(2,6-Dichloro-phenyl)-3H-benzoimidazole-5-carboxylic acid (3-chloro-4-fluoro-phenyl)-amide
2-(2,6-Dichloro-phenyl)-3H-benzoimidazole-5-carboxylic acid (3,4-dichloro-phenyl)-amide
2-(2,6-Dichloro-phenyl)-3H-benzoimidazole-5-carboxylic acid (4-fluoro-3-methyl-phenyl)-amide
2-(2,6-Dichloro-phenyl)-3H-benzoimidazole-5-carboxylic acid (3-chloro-4-methyl-phenyl)-amide
2-(2,6-Dichloro-phenyl)-3H-benzoimidazole-5-carboxylic acid (3,4-difluoro-phenyl)-amide
2-(2,6-Dichloro-phenyl)-3H-benzoimidazole-5-carboxylic acid (3,4-dimethoxy-phenyl)-amide
2-(2,6-Dichloro-phenyl)-3H-benzoimidazole-5-carboxylic acid (1H-indazol-5-yl)-amide
2-(2,6-Dichloro-phenyl)-3H-benzoimidazole-5-carboxylic acid (1H-indazol-6-yl)-amide
2-(2,6-Dichloro-phenyl)-3H-benzoimidazole-5-carboxylic acid (2-methyl-benzothiazol-6-yl)-amide
2-(2,6-Dichloro-phenyl)-3H-benzoimidazole-5-carboxylic acid (2-methyl-benzothiazol-5-yl)-amide
2-(2,6-Dichloro-phenyl)-3H-benzoimidazole-5-carboxylic acid quinoli6-ylamide
2-(2,6-Dichloro-phenyl)-1H-benzoimidazole-5-carboxylic acid pyridin-2-ylamide
2-(2,6-Dichloro-phenyl)-1H-benzoimidazole-5-carboxylic acid (6-chloro-pyridin-2-yl)-amide
2-(2,6-Dichloro-phenyl)-1H-benzoimidazole-5-carboxylic acid (6-methyl-pyridin-2-yl)-amide
2-(2,6-Dichloro-phenyl)-1H-benzoimidazole-5-carboxylic acid quinoxalin-6-ylamide
2-(2,6-Dichloro-phenyl)-1H-benzoimidazole-5-carboxylic acid (6-chloro-pyridin-3-yl)-amide
2-(2,6-Dichloro-phenyl)-1H-benzoimidazole-5-carboxylic acid pyridin-3-ylamide
2-(2,6-Dichloro-phenyl)-1H-benzoimidazole-5-carboxylic acid (5-chloro-pyridin-2-yl)-amide
2-(2,6-Dichloro-phenyl)-1H-benzoimidazole-5-carboxylic acid (5-methyl-pyridin-2-yl)-amide
2-(2,6-Dichloro-phenyl)-1H-benzoimidazole-5-carboxylic acid (4-methyl-pyridin-2-yl)-amide
2-(2,6-Dichloro-phenyl)-1H-benzoimidazole-5-carboxylic acid (6-chloro-pyridazin-3-yl)-amide
2-(2,6-Dichloro-phenyl)-1H-benzoimidazole-5-carboxylic acid pyrazin-2-ylamide
2-(2,6-Dichloro-phenyl)-1H-benzoimidazole-5-carboxylic acid (4-methyl-pyrimidin-2-yl)-amide
2-(2,6-Dichloro-phenyl)-1H-benzoimidazole-5-carboxylic acid pyridazin-3-ylamide
2-(2,6-Dichloro-phenyl)-1H-benzoimidazole-5-carboxylic acid (6-chloro-pyrazin-2-yl)-amide
2-(2,6-Dichloro-phenyl)-1H-benzoimidazole-5-carboxylic acid (5-chloro-pyrimidin-2-yl)-amide
2-(2,6-Dichloro-phenyl)-1H-benzoimidazole-5-carboxylic acid pyrimidin-4-ylamide
2-(2,6-Dichloro-phenyl)-1H-benzoimidazole-5-carboxylic acid [3-(2H-tetrazol-5-yl)-phenyl]-amide
2-(2,6-Dichloro-phenyl)-3H-benzoimidazole-5-carboxylic acid [2-(3-chloro-phenyl)-ethyl]-amide
2-(2,6-Dichloro-phenyl)-3H-benzoimidazole-5-carboxylic acid [2-(4-bromo-phenyl)-ethyl]-amide
2-(2,6-Dichloro-phenyl)-3H-benzoimidazole-5-carboxylic acid [2-(4-fluoro-phenyl)-ethyl]-amide
2-(2,6-Dichloro-phenyl)-3H-benzoimidazole-5-carboxylic acid [2-(3,4-dimethyl-phenyl)-ethyl]-amide
2-(2,6-Dichloro-phenyl)-3H-benzoimidazole-5-carboxylic acid [2-(3-ethoxy-phenyl)-ethyl]-amide
2-(2,6-Dichloro-phenyl)-3H-benzoimidazole-5-carboxylic acid [2-(4-methoxy-phenyl)-ethyl]-amide
2-(2,6-Dichloro-phenyl)-3H-benzoimidazole-5-carboxylic acid [2-(2-methoxy-phenyl)-ethyl]-amide
2-(2,6-Dichloro-phenyl)-3H-benzoimidazole-5-carboxylic acid [2-(3-fluoro-phenyl)-ethyl]-amide
2-(2,6-Dichloro-phenyl)-3H-benzoimidazole-5-carboxylic acid [2-(2,4-dichloro-phenyl)-ethyl]-amide
2-(2,6-Dichloro-phenyl)-3H-benzoimidazole-5-carboxylic acid [2-(2-ethoxy-phenyl)-ethyl]-amide
2-(2,6-Dichloro-phenyl)-3H-benzoimidazole-5-carboxylic acid [2-(4-ethyl-phenyl)-ethyl]-amide
2-(2,6-Dichloro-phenyl)-3H-benzoimidazole-5-carboxylic acid [2-(2,4-dimethyl-phenyl)-ethyl]-amide
2-(2,6-Dichloro-phenyl)-3H-benzoimidazole-5-carboxylic acid ((R)-2-phenyl-propyl)-amide
2-(2,6-Dichloro-phenyl)-3H-benzoimidazole-5-carboxylic acid [2-(3,4-dimethoxy-phenyl)-ethyl]-amide
2-(2,6-Dichloro-phenyl)-3H-benzoimidazole-5-carboxylic acid [2-(3-bromo-4-methoxy-phenyl)-ethyl]-amide
2-(2,6-Dichloro-phenyl)-3H-benzoimidazole-5-carboxylic acid [2-(2-fluoro-phenyl)-ethyl]-amide
2-(2,6-Dichloro-phenyl)-3H-benzoimidazole-5-carboxylic acid [2-(2,5-dimethoxy-phenyl)-ethyl]-amide
2-(2,6-Dichloro-phenyl)-3H-benzoimidazole-5-carboxylic acid [2-(4-phenoxy-phenyl)-ethyl]-amide
2-(2,6-Dichloro-phenyl)-3H-benzoimidazole-5-carboxylic acid [2-(4-ethoxy-3-methoxy-phenyl)-ethyl]-amide
2-(2,6-Dichloro-phenyl)-3H-benzoimidazole-5-carboxylic acid [2-(4-ethoxy-phenyl)-ethyl]-amide
2-(2,6-Dichloro-phenyl)-3H-benzoimidazole-5-carboxylic acid [2-(2,6-dichloro-phenyl)-ethyl]-amide
2-(2,6-Dichloro-phenyl)-3H-benzoimidazole-5-carboxylic acid [2-(4-hydroxy-phenyl)-ethyl]-amide
2-(2,6-Dichloro-phenyl)-3H-benzoimidazole-5-carboxylic acid [2-(2,5-dimethyl-phenyl)-ethyl]-amide
2-(2,6-Dichloro-phenyl)-3H-benzoimidazole-5-carboxylic acid (5-chloro-benzo[b]thiophen-3-ylmethyl)-amide
2-(2,6-Dichloro-phenyl)-1H-benzoimidazole-5-carboxylic acid (2-pyridin-2-yl-ethyl)-amide
2-(2,6-Dichloro-phenyl)-1H-benzoimidazole-5-carboxylic acid (2-pyridin-3-yl-ethyl)-amide
2-(2,6-Dichloro-phenyl)-1H-benzoimidazole-5-carboxylic acid (2-pyridin-4-yl-ethyl)-amide
2-(2,6-Dichloro-phenyl)-3H-benzoimidazole-5-carboxylic acid (3-chloro-phenyl)-methyl-amide
2-(2,6-Dichloro-phenyl)-3H-benzoimidazole-5-sulfonic acid (3,4-dimethyl-phenyl)-amide
2-(2,6-Dichloro-phenyl)-3H-benzoimidazole-5-sulfonic acid (2-methyl-benzothiazolyl-5-yl)-amide
2-(2,6-Dimethyl-phenyl)-3H-benzoimidazole-5-carboxylic acid (3-chloro-phenyl)-amide
2-o-Tolyl-3H-benzoimidazole-5-carboxylic acid (3-chloro-phenyl)-amide
{4-[6-(3-Chloro-phenylcarbamoyl)-1H-benzoimidazol-2-yl]-3,5-dimethyl-phenoxy}-acetic acid ethyl ester
{4-[6-(3-Chloro-phenylcarbamoyl)-1H-benzoimidazol-2-yl]-3,5-dimethyl-phenoxy}-acetic acid
{4-[6-(3-Chloro-phenylcarbamoyl)-1H-benzoimidazol-2-yl]-3-methyl-phenyl}-carbamic acid ethyl ester
2-Phenyl-3H-benzoimidazole-5-carboxylic acid (3-chloro-phenyl)-amide
2-(2-Chloro-phenyl)-3H-benzoimidazole-5-carboxylic acid (3-chloro-phenyl)-amide
2-(3-Chloro-phenyl)-3H-benzoimidazole-5-carboxylic acid (3-chloro-phenyl)-amide
2-(4-Chloro-phenyl)-3H-benzoimidazole-5-carboxylic acid (3-chloro-phenyl)-amide
2-(2-Chloro-6-nitro-phenyl)-3H-benzoimidazole-5-carboxylic acid (3-chloro-phenyl)-amide
2-(2-Methoxy-naphthalen-1-yl)-3H-benzoimidazole-5-carboxylic acid (3-chloro-phenyl)-amide
2-(2-Methoxy-phenyl)-3H-benzoimidazole-5-carboxylic acid (3-chloro-phenyl)-amide
2-(2-Trifluoromethyl-phenyl)-3H-benzoimidazole-5-carboxylic acid (3-chloro-phenyl)-amide
2-(2-Fluoro-phenyl)-3H-benzoimidazole-5-carboxylic acid (3-chloro-phenyl)-amide
2-(2-Cyano-phenyl)-3H-benzoimidazole-5-carboxylic acid (3-chloro-phenyl)-amide
2-(2-Chloro-6-fluoro-phenyl)-3H-benzoimidazole-5-carboxylic acid (3-chloro-phenyl)-amide
2-(2,3-Dichloro-phenyl)-3H-benzoimidazole-5-carboxylic acid (3-chloro-phenyl)-amide
2-(2,5-Dichloro-phenyl)-3H-benzoimidazole-5-carboxylic acid (3-chloro-phenyl)-amide
2-(2,4-Dichloro-phenyl)-3H-benzoimidazole-5-carboxylic acid (3-chloro-phenyl)-amide
2-(4-Methoxy-naphthalen-1-yl)-3H-benzoimidazole-5-carboxylic acid (3-chloro-phenyl)-amide
2-(4-Acetylamino-phenyl)-3H-benzoimidazole-5-carboxylic acid (3-chloro-phenyl)-amide
2-(3-Phenoxy-phenyl)-3H-benzoimidazole-5-carboxylic acid (3-chloro-phenyl)-amide
2-Naphthalen-1-yl-3H-benzoimidazole-5-carboxylic acid (3-chloro-phenyl)-amide
4-[6-(3-Chloro-phenylcarbamoyl)-1H-benzoimidazol-2-yl]-benzoic acid methyl ester
2-(4-Cyano-phenyl)-3H-benzoimidazole-5-carboxylic acid (3-chloro-phenyl)-amide
2-(2,6-Dimethoxy-phenyl)-3H-benzoimidazole-5-carboxylic acid (3-chloro-phenyl)-amide
2-(4-tert-Butyl-phenyl)-3H-benzoimidazole-5-carboxylic acid (3-chloro-phenyl)-amide
2-(2,6-Dinitro-phenyl)-3H-benzoimidazole-5-carboxylic acid (3-chloro-phenyl)-amide
2-(2,6-Difluoro-phenyl)-3H-benzoimidazole-5-carboxylic acid (3-chloro-phenyl)-amide
2-(2-Fluoro-6-methoxy-phenyl)-3H-benzoimidazole-5-carboxylic acid (3-chloro-phenyl)-amide
2-(2-Fluoro-6-trifluoromethyl-phenyl)-3H-benzoimidazole-5-carboxylic acid (3-chloro-phenyl)-amide
2-(2-Chloro-6-methanesulfonylamino-phenyl)-3H-benzoimidazole-5-carboxylic acid (3-chloro-phenyl)-amide
2-(2-Acetylamino-6-chloro-phenyl)-3H-benzoimidazole-5-carboxylic acid (3-chloro-phenyl)-amide
4-[6-(3-Chloro-phenylcarbamoyl)-1H-benzoimidazol-2-yl]-3-methyl-benzoic acid
4-[6-(3-Chloro-phenylcarbamoyl)-1H-benzoimidazol-2-yl]-3-methyl-benzoic acid methyl ester
2-(4-Acetylamino-2,6-dimethyl-phenyl)-3H-benzoimidazole-5-carboxylic acid (3,4-dimethyl-phenyl)-amide
2-[2,6-dimethyl-4-(2-oxo-2-pyrrolidin-1-yl-ethoxy)-phenyl]-3H-benzoimidazole-5-carboxylic acid (3,4-dimethyl-phenyl)-amide
Toluene-4-sulfonic acid 4-[6-(3,4-dimethyl-phenylcarbamoyl)-1H-benzoimidazol-2-yl]-3,5-dimethyl-phenyl ester
2-[2,6-dimethyl-4-(2-pyrrolidin-1-yl-ethoxy)-phenyl]-3H-benzoimidazole-5-carboxylic acid (3,4-dimethyl-phenyl)-amide
2-[2,6-Dimethyl-4-(1H-tetrazol-5-yl-methoxy)-phenyl]-3H-benzoimidazole-5-carboxylic acid (3,4-dimethyl-phenyl)-amide
{4-[6-(3,4-Dimethyl-phenylcarbamoyl)-1H-benzoimidazol-2-yl]-3,5-dimethyl-phenoxy}-acetic acid ethyl ester
2-(4-Cyano-2,6-dimethyl-phenyl)-3H-benzoimidazole-5-carboxylic acid (3,4-dimethyl-phenyl)-amide
Trifluoro-methanesulfonic acid 4-[6-(3,4-dimethyl-phenylcarbamoyl)-1H-benzoimidazol-2-yl]-3,5-dimethyl-phenyl ester
2-(2,6-Dimethyl-phenyl)-3H-benzoimidazole-5-carboxylic acid (3,4-dimethyl-phenyl)-amide
2-(4-Hydroxy-2,6-dimethyl-phenyl)-3H-benzoimidazole-5-carboxylic acid (3,4-dimethyl-phenyl)-amide
2-(4-Methoxy-2,6-dimethyl-phenyl)-3H-benzoimidazole-5-carboxylic acid (3,4-dimethyl-phenyl)-amide
2-(4-Carbamoylmethoxy-2,6-dimethyl-phenyl)-3H-benzoimidazole-5-carboxylic acid
(3,4-dimethyl-phenyl)-amide
2-(2,6-Dimethyl-4-methylcarbamoylmethoxy-phenyl)-3H-benzoimidazole-5-carboxylic acid (3,4-dimethyl-phenyl)-amide
2-(4-Dimethylcarbamoylmethoxy-2,6-dimethyl-phenyl)-3H-benzoimidazole-5-carboxylic acid (3,4-dimethyl-phenyl)-amide
Methanesulfonic acid 4-[6-(3,4-dimethyl-phenylcarbamoyl)-1H-benzoimidazol-2-yl]-3,5-dimethyl-phenyl ester
{4-[6-(3,4-Dimethyl-phenylcarbamoyl)-1H-benzoimidazol-2-yl]-3,5-dimethyl-phenoxy}-acetic acid
2-{2,6-Dimethyl-4-[2-(4-methyl-piperazin-1-yl)-2-oxo-ethoxy]-phenyl}-3H benzoimidazole-5-carboxylic acid (3,4-dimethyl-phenyl)-amide
4-[6-(3,4-Dimethyl-phenylcarbamoyl)-1H-benzoimidazol-2-yl]-3,5-dimethyl-benzoic acid
2-[2,6-dimethyl-4-(4-methyl-piperazine-1-carbonyl)-phenyl]-3H-benzoimidazole-5-carboxylic acid (3,4-dimethyl-phenyl)-amide
2-[2,6-Dimethyl-4-(2H-tetrazol-5-yl)-phenyl]-3H-benzoimidazole-5-carboxylic acid (3,4-dimethyl-phenyl)-amide
[2-(2,6-Dichloro-phenyl)-3H-benzoimidazol-5-ylmethyl]-(3,4-dimethyl-phenyl)-amine
2-(4-Carbamoylmethoxy-2,6-dimethyl-phenyl)-3H-benzoimidazole-5-carboxylic acid (2-methyl-benzothiazol-5-yl)-amide
2-(2,6-Dimethyl-4-methylcarbamoylmethoxy-phenyl)-3H-benzoimidazole-5-carboxylic acid (2-methyl-benzothiazol-5-yl)-amide
2-(4-Dimethylcarbamoylmethoxy-2,6-dimethyl-phenyl)-3H-benzoimidazole-5-carboxylic acid (2-methyl-benzothi azol-5-yl)-amide
2-[2,6-Dimethyl-4-(2-oxo-2-pyrrolidin-1-yl-ethoxy)-phenyl]-3H-benzoimi dazole-5-carboxylic acid (2-methyl-benzothiazol-5-yl)-amide
Trifluoro-methanesulfonic acid 3,5-dimethyl-4-[6-(2-methyl-benzothiazol-5-ylcarbamoyl)-1H-benzoimidazol-2-yl]-phenyl ester
Methanesulfonic acid 3,5-dimethyl-4-[6-(2-methyl-benzothiazol-5-ylcarb amoyl)-1H-benzoimidazol-2-yl]-phenyl ester
Toluene-4-sulfonic acid 3,5-dimethyl-4-[6-(2-methyl-benzothiazol-5-ylcarbamoyl)-1H-benzoimidazol-2-yl]-phenyl ester
{3,5-Dimethyl-4-[6-(2-methyl-benzothiazol-5-ylcarbamoyl)-1H-benzoimidazol-2-yl]-phenoxy}-acetic acid ethyl ester
{3,5-dimethyl-4-[6-(2-methyl-benzothiazol-5-ylcarbamoyl)-1H-benzoimidazol-2-yl]-phenoxy}-acetic acid
2-(2,6-Dichlorophenyl)-3H-benzoimidazole-5-carboxylic acid (3,5-dimethoxyphenyl)-amide
2-(2,6-Dichlorophenyl)-3H-benzoimidazole-5-carboxylic acid benzo[1,3]dioxol-5-ylamide
2-(2,6-Dichlorophenyl)-3H-benzoimidazole-5-carboxylic acid (3-chloro-4-methoxyphenyl)-amide
2-(2,6-Dichlorophenyl)-3H-benzoimidazole-5-carboxylic acid (3-trifluoromethylphenyl)-amide
2-(2,6-Dichlorophenyl)-3H-benzoimidazole-5-carboxylic acid (4-trifluoromethylphenyl)-amide
2-(2,6-Dichlorophenyl)-3H-benzoimidazole-5-carboxylic acid (3-trifluoromethoxyphenyl)-amide
2-(2,6-Dichlorophenyl)-3H-benzoimidazole-5-carboxylic acid (4-fluoro-3-trifluoromethylphenyl)-amide
2-(2,6-Dichlorophenyl)-3H-benzoimidazole-5-carboxylic acid (3,5-difluorophenyl)-amide
2-(2,6-Dichlorophenyl)-3H-benzoimidazole-5-carboxylic acid (4-nitrophenyl)-amide
2-(2,6-Dichlorophenyl)-3H-benzoimidazole-5-carboxylic acid (2,4-dichlorophenyl)-amide
2-(2,6-Dichlorophenyl)-3H-benzoimidazole-5-carboxylic acid (3,5-dichlorophenyl)-amide
2-(2,6-Dichlorophenyl)-3H-benzoimidazole-5-carboxylic acid (2-fluorophenyl)-amide
2-(2,6-Dichlorophenyl)-3H-benzoimidazole-5-carboxylic acid (4-chloro-2-fluorophenyl)-amide
2-(2,6-Dichlorophenyl)-3H-benzoimidazole-5-carboxylic acid biphenyl-4-ylamide
2-(2,6-Dichlorophenyl)-3H-benzoimidazole-5-carboxylic acid (4-phenoxyphenyl)-amide
2-(2,6-Dichlorophenyl)-3H-benzoimidazole-5-carboxylic acid (2-methoxyphenyl)-amide
2-(2,6-Dichlorophenyl)-3H-benzoimidazole-5-carboxylic acid (4-methanesulfonylphenyl)-amide
2-(2,6-Dichlorophenyl)-3H-benzoimidazole-5-carboxylic acid m-tolylamide
2-(2,6-Dichlorophenyl)-3H-benzoimidazole-5-carboxylic acid (3-phenoxyphenyl)-amide
2-(2,6-Dichlorophenyl)-3H-benzoimidazole-5-carboxylic acid (3-cyano-4-methylphenyl)-amide
2-(2,6-Dichlorophenyl)-3H-benzoimidazole-5-carboxylic acid (4-tert-butylphenyl)-amide
2-(2,6-Dichlorophenyl)-3H-benzoimidazole-5-carboxylic acid (3,5-di-tert-butylphenyl)-amide
3-{[2-(2,6-Dichlorophenyl)-3H-benzoimidazole-5-carbonyl]-amino}-benzoic acid methyl ester
2-(2,6-Dichlorophenyl)-3H-benzoimidazole-5-carboxylic acid (3-dimethylaminophenyl)-amide
2-(2,6-Dichlorophenyl)-3H-benzoimidazole-5-carboxylic acid (3-phenylpropyl)-amide
2-(2,6-Dichlorophenyl)-3H-benzoimidazole-5-carboxylic acid (3-oxazol-5-yl-phenyl)-amide
2-(2,6-Dichlorophenyl)-3H-benzoimidazole-5-carboxylic acid (4-oxazol-5-yl-phenyl)-amide
2-(2,6-Dichlorophenyl)-3H-benzoimidazole-5-carboxylic acid naphthalen-2-ylamide
2-(2,6-Dichlorophenyl)-3H-benzoimidazole-5-carboxylic acid (5-oxo-5,6,7,8-tetrahydronaphthalen-2-yl)-amide
2-(2,6-Dichlorophenyl)-3H-benzoimidazole-5-carboxylic acid indan-5-ylamide
2-(2,6-Dichlorophenyl)-3H-benzoimidazole-5-carboxylic acid (2-oxo-4-trifluoromethyl-2H-chromen-7-yl)-amide
2-(2,6-Dichlorophenyl)-3H-benzoimidazole-5-carboxylic acid (4-methylthiazol-2-yl)-amide
2-(2,6-Dichlorophenyl)-3H-benzoimidazole-5-carboxylic acid (4,5-dimethylthiazol-2-yl)-amide
2-(2,6-Dichlorophenyl)-3H-benzoimidazole-5-carboxylic acid (5,6,7,8-tetrahydronaphthalen-2-yl)-amide
2-(2,6-Dichlorophenyl)-3H-benzoimidazole-5-carboxylic acid (8-oxo-5,6,7,8-tetrahydronaphthalen-2-yl)-amide
2-(2,6-Dichlorophenyl)-3H-benzoimidazole-5-carboxylic acid (8-hydroxy-5,6,7,8-tetrahydronaphthalen-2-yl)-amide
2-(2,6-Dichlorophenyl)-3H-benzoimidazole-5-carboxylic acid (4-phenylbutyl)-amide
2-(2,6-Dichlorophenyl)-3H-benzoimidazole-5-carboxylic acid endo-bicyclo[2.2.1]hept-2-ylamide
2-(2,6-Dichlorophenyl)-3H-benzoimidazole-5-carbokylic acid exo-bicyclo[2.2.1]hept-2-ylamide
2-(2,6-Dichlorophenyl)-3H-benzoimidazole-5-carboxylic acid adamantan-2-ylamide
2-(2,6-Dichlorophenyl)-3H-benzoimidazole-5-carboxylic acid (4-methyl-2-oxo-2H-chromen-7-yl)-amide
2-(2,6-Dichlorophenyl)-3H-benzoimidazole-5-carboxylic acid (2,3-dihydrobenzo[1,4]dioxin-6-yl)-amide
2-(2,6-Dichlorophenyl)-3H-benzoimidazole-5-carboxylic acid [2-(pyrrolidine-1-carbonyl)-phenyl]-amide
2-(2,6-Dichlorophenyl)-3H-benzoimidazole-5-carboxylic acid (4-butylphenyl)-amide
2-(2,6-Dichlorophenyl)-3H-benzoimidazole-5-carboxylic acid (4-cyclohexylphenyl)-amide
2-(2,6-Dichlorophenyl)-3H-benzoimidazole-5-carboxylic acid (4-tert-butylcyclohexyl)-amide
2-(2,6-Dichlorophenyl)-3H-benzoimidazole-5-carboxylic acid quinolin-7-ylamide
2-(2,6-Dichlorophenyl)-3H-benzoimidazole-5-carboxylic acid isoquinolin-3-ylamide
2-(2,6-Dichlorophenyl)-3H-benzoimidazole-5-carboxylic acid (2-methylquinolin-6-yl)-amide
2-(2,6-Dichlorophenyl)-3H-benzoimidazole-5-carboxylic acid (4-methoxynaphthalen-2-yl)-amide
2-(2,6-Dichlorophenyl)-3H-benzoimidazole-5-carboxylic acid quinolin-3-ylamide
2-(2,6-Dichlorophenyl)-3H-benzoimidazole-5-carboxylic acid (4-methoxymethyl-2-oxo-2H-chromen-7-yl)-amide
2-(2,6-Dichlorophenyl)-3H-benzoimidazole-5-carboxylic acid quinolin-2-ylamide
2-(2,6-Dichlorophenyl)-3H-benzoimidazole-5-carboxylic acid anthracen-2-ylamide.
(E)-3-(4-{[2-(2,6-Dichlorophenyl)-3H-benzoimidazole-5-carbonyl]-amino}-phenyl)-acrylic acid ethyl ester
2-(2,6-Dichlorophenyl)-3H-benzoimidazole-5-carboxylic acid (4-ethylphenyl)-amide
2-(2,6-Dichlorophenyl)-3H-benzoimidazole-5-carboxylic acid (4-isopropylphenyl)-amide
2-(2,6-Dichlorophenyl)-3H-benzoimidazole-5-carboxylic acid (2,6-dimethoxyphenyl)-amide
2-(2,6-Dichlorophenyl)-3H-benzoimidazole-5-carboxylic acid (2,5-di-tert-butylphenyl)-amide
2-(2,6-Dichlorophenyl)-3H-benzoimidazole-5-carboxylic acid (2,6-diisopropylphenyl)-amide
2-(2,6-Dichlorophenyl)-3H-benzoimidazole-5-carboxylic acid (3-phenylcarbamoylphenyl)-amide
2-(2,6-Dichlorophenyl)-3H-benzoimidazole-5-carboxylic acid [2-(4-fluorophenoxy)-pyridin-3-yl]-amide
2-(2,6-Dichlorophenyl)-3H-benzoimidazole-5-carboxylic acid (4-chloro-3′-trifluoromethylphenyl)-amide
2-(2,6-Dichlorophenyl)-3H-benzoimidazole-5-carboxylic acid (4-sec-butylphenyl)-amide
2-(2,6-Dichlorophenyl)-3H-benzoimidazole-5-carboxylic acid (2-phenyl-2H-pyrazol-3-yl)-amide
2-(2,6-Dichlorophenyl)-3H-benzoimidazole-5-carboxylic acid (2-methyl-5-phenyl-2H-pyrazol-3-yl)-amide
2-(2,6-Dichlorophenyl)-3H-benzoimidazole-5-carboxylic acid (8-hydroxyquinolin-2-yl)-amide
2-(2,3-Dimethylphenyl)-3H-benzoimidazole-5-carboxylic acid (3,4-dimethylphenyl)-amide
2-(2,6-Dimethylphenyl)-3H-benzoimidazole-5-carboxylic acid naphthalen-2-ylamide
2-(2,6-Dimethylphenyl)-3H-benzoimidazole-5-carboxylic acid (4-methyl-2-oxo-2H-chromen-7-yl)-amide
2-(4-Chloro-phenyl)-3-(2-hydroxy-ethyl)-3H-benzoimidazole-5-carboxylic acid (3,4-dimethyl-phenyl)-amide
2-(2,6-Dichloro-phenyl)-3-(2-hydroxy-ethyl)-3H-benzoimidazole-5-carboxylic acid (3,4-dimethyl-phenyl)-amide
2-(2-Chloro-6-nitro-phenyl)-3-(2-hydroxy-ethyl)-3H-benzoimidazole-5-carboxylic acid
(3,4-dimethyl-phenyl)-amide
2-(2,6-Dimethyl-phenyl)-3-(2-hydroxy-ethyl)-3H-benzoimidazole-5-carboxylic acid (3,4-dimethyl-phenyl)-amide
2-(2,6-Dimethoxy-phenyl)-3-(2-hydroxy-ethyl)-3H-benzoimidazole-5-carboxylic acid
(3,4-dimethyl-phenyl)-amide
2-(2-Chloro-phenyl)-3-(2-hydroxy-ethyl)-3H-benzoimidazole-5-carboxylic acid (3,4-dimethyl-phenyl)-amide
2-(4-Chloro-phenyl)-3-methyl-3H-benzoimidazole-5-carboxylic acid (3,4-dimethyl-phenyl)-amide
2-(2,6-Dichloro-phenyl)-3-methyl-3H-benzoimidazole-5-carboxylic acid (3,4-dimethyl-phenyl)-amide
2-(2-Chloro-6-nitro-phenyl)-3-methyl-3H-benzoimidazole-5-carboxylic acid (3,4-dimethyl-phenyl)-amide
2-(2,6-Dimethyl-phenyl)-3-methyl-3H-benzoimidazole-5-carboxylic acid (3,4-dimethyl-phenyl)-amide
2-(2,6-Dimethoxy-phenyl)-3-methyl-3H-benzoimidazole-5-carboxylic acid (3,4-dimethyl-phenyl)-amide
2-(2-Chloro-phenyl)-3-methyl-3H-benzoimidazole-5-carboxylic acid (3,4-dimethyl-phenyl)-amide
2-(2,6-Dichlorophenyl)-1-methyl-1H-benzoimidazole-5-carboxylic acid (3,4-dimethylphenyl)-amide
2-(2,6-dichloro-phenyl)-1H-indole-5-carboxylic acid (2-methyl-benzothiazol-5-yl)-amide
2-(2,6-Dichloro-phenyl)-1H-indole-5-carboxylic acid (3,4-dimethyl-phenyl)-amide
2-(2,6-Dichloro-phenyl)-1H-indole-5-carboxylic acid (3-chloro-phenyl)-amide
2-(2,6-dichloro-phenyl)-benzooxazole-6-carboxylic acid (2-o-tolyl-ethyl)-amide
2-(2,6-Dichloro-phenyl)-benzooxazole-6-carboxylic acid (3-chloro-phenyl)-amide
2-(2,6-Dichloro-phenyl)-benzooxazole-6-carboxylic acid (3,4-dimethyl-phenyl)-amide
2-(2,6-Dichloro-phenyl)-benzooxazole-6-carboxylic acid (3,5-dimethyl-phenyl)-amide
2-(2,6-Dichloro-phenyl)-benzooxazole-6-carboxylic acid p-tolylamide
2-(2,6-Dichloro-phenyl)-benzooxazole-6-carboxylic acid (3-chloro-4-methyl-phenyl)-amide
2-(2,6-Dichloro-phenyl)-benzooxazole-6-carboxylic acid (4-fluoro-3-methyl-phenyl)-amide
2-(2,6-Dichloro-phenyl)-benzooxazole-6-carboxylic acid (2-methyl-benzothiazol-6-yl)-amide
2-(2,6-Dichloro-phenyl)-benzooxazole-6-carboxylic acid (1H-indazol-5-yl)-amide
2-(2,6-Dichloro-phenyl)-benzooxazole-6-carboxylic acid (1H-indazol-6-yl)-amide
2-(2,6-Dichloro-phenyl)-benzooxazole-6-carboxylic acid [2-(2-methoxy-phenyl)-ethyl]-amide
2-(2,6-Dichloro-phenyl)-benzooxazole-6-carboxylic acid [2-(3-fluoro-phenyl)-ethyl]-amide
2-(2,6-Dichloro-phenyl)-benzooxazole-6-carboxylic acid [2-(4-fluoro-phenyl)-ethyl]-amide
2-(2,6-Dichloro-phenyl)-benzooxazole-6-carboxylic acid benzylamide
2-(2,6-Dichloro-phenyl)-benzooxazole-6-carboxylic acid 2-methyl-benzylamide
2-(2,6-Dichloro-phenyl)-benzooxazole-6-carboxylic acid 2-chloro-benzylamide
2-(2,6-Dichloro-phenyl)-benzooxazole-6-carboxylic acid 3-methoxy-benzylamide
2-(2,6-Dichloro-phenyl)-benzooxazole-6-carboxylic acid 4-methoxy-benzylamide
2-(2,6-Dichlorophenyl)-benzooxazole-5-carboxylic acid (3,4-dimethylphenyl)-amide
2-(2,6-Dichlorophenyl)-benzooxazole-5-carboxylic acid (2-methylbenzothiazol-5-yl)-amide
2-(2,6-Dichlorophenyl)-benzooxazole-5-carboxylic acid [2-(4-ethylphenyl)-ethyl]-amide
2-(2,6-Dichlorophenyl)-benzooxazole-5-carboxylic acid (3-phenylpropyl)-amide
2-(4-Dimethylcarbamoylmethoxy-2,6-dimethylphenyl)-benzooxazole-5-carboxylic acid
(3,4-dimethylphenyl)-amide
{4-[5-(3,4-Dimethyl-phenylcarbamoyl)-1H-benzoimidazol-2-yl]-3,5-dimethyl-phenylamino}-acetic acid methyl ester
{4-[5-(3,4-Dimethylphenylcarbamoyl)-1H-benzoimidazol-2-yl]-3,5-dimethylphenylamino}-acetic acid
2-[4-(2-Hydroxyethylamino)-2,6-dimethylphenyl]-1H-benzoimidazole-5-carboxylic acid
(3,4-dimethyl-phenyl)-amide
3-{4-[5-(3,4-Dimethylphenylcarbamoyl)-1H-benzoimidazol-2-yl]-3,5-dimethylphenyl}-propionic acid tert-butyl ester
3-{4-[5-(3,4-dimethylphenylcarbamoyl)-1H-benzoimidazol-2-yl]-3,5-dimethylphenyl}-propionic acid
2-(2,6-Dimethylphenyl)-1,3-dioxo-2,3-dihydro-1H-isoindole-5-carboxylic acid (3,4-dimethylphenyl)-amide
2-(2,6-Dichlorophenyl)-1,3-dioxo-2,3-dihydro-1H-isoindole-5-carboxylic acid (3,4-dimethylphenyl)-amide
2-(2,6-Dichlorophenyl)-3H-benzoimidazole-5-carboxylic acid isoquinolin-1-ylamide
2-(2,6-Dichlorophenyl)-3H-benzoimidazole-5-carboxylic acid (4-vinylphenyl)-amide
2-(2,6-Dichlorophenyl)-3H-benzoimidazole-5-carboxylic acid (4-cyanophenyl)-amide
3-(4-{[2-(2,6-Dichlorophenyl)-3H-benzoimidazole-5-carbonyl]-amino}-phenyl)-propionic acid
3-(4-{[2-(2,6-Dichlorophenyl)-3H-benzoimidazole-5-carbonyl]-amino}-phenyl)-propionic acid ethyl ester
2-(2,6-Dichlorophenyl)-3H-benzoimidazole-5-carboxylic acid (1,1-dimethylindan-5-yl)-amide
2-(2,6-Dichlorophenyl)-3H-benzimidazole-5-carboxylic acid decylamide
2-(2,6-Dichlorophenyl)-3H-benzimidazole-5-carboxylic acid [2-(4-tert-butylphenyl)-ethyl]amide
2-(2-Chloro-6-methylphenyl)-3H-benzoimidazole-5-carboxylic acid (3,4-dimethylphenyl)-amide
2-(2-Chloro-6-trifluoromethylphenyl)-3H-benzoimidazole-5-carboxylic acid quinolin-2-ylamide
2-(2,4-Dichloro-6-methoxyphenyl)-3H-benzoimidazole-5-carboxylic acid quinolin-2-ylamide
2-(3,5-Dichloro-pyridin-4-yl)-3H-benzoimidazole-5-carboxylic acid quinolin-2-ylamide
2-(2,6-Dichlorophenyl)-3H-benzoimidazole-5-carboxylic acid [2-(4-chlorophenyl)-2-oxo-ethyl]-amide
2-(2,6-Dichlorophenyl)-3H-benzimidazole-5-carboxylic acid [2-(4-chloro-phenyl)-1-methyl-2-oxoethyl]-amide
2-(2,6-Dichlorophenyl)-6,7-difluoro-3H-benzoimidazole-5-carboxylic acid quinolin-2-ylamide
N-[2-(2,6-Dichlorophenyl)-3H-benzoimidazol-5-yl]-3,4-dimethylbenzamide
Quinoline-2-carboxylic acid [2-(2,6-dichlorophenyl)-3H-benzimidazol-5-yl]-amide
2-(2,6-Dimethylphenyl)-3H-benzimidazole-5-carboxylic acid (4-tert-butylphenyl)-amide
1-[2-(2,6-Dichlorophenyl)-3H-benzimidazol-5-yl]-3-(3,4-dimethylphenyl)-urea
2-(2,4,6-Trichlorophenyl)-3H-benzoimidazole-5-carboxylic acid quinolin-2-ylamide
2-(2,6-Dimethylphenyl)-1H-indole-6-carboxylic acid (4-tert-butylphenyl)-amide
2-(2,6-Dichlorophenyl)-1H-indole-6-carboxylic acid (3,4-dimethylphenyl)-amide
2-(2,6-Dichlorophenyl)-1H-indole-6-carboxylic acid quinolin-2-ylamide
2-(2,6-Dimethylphenyl)-1H-indole-6-carboxylic acid quinolin-2-ylamide
2-(2,6-Dichlorophenyl)-1H-indole-6-carboxylic acid (6-trifluoromethylpyridin-3-yl)-amide
2-(2,6-Dichlorophenyl)-1-ethoxy-1H-indole-6-carboxylic acid (3,4-dimethylphenyl)-amide
2-(2,6-Dimethylphenyl)-1H-indole-6-carboxylic acid (3,4-dimethylphenyl)-amide
2-(2,6-Dichlorophenyl)-1H-indole-6-carboxylic acid thiazolo[5,4-b]pyridin-2-ylamide
2-(2,6-Dichlorophenyl)-1H-indole-6-carboxylic acid (5-bromothiazolo[5,4-b]pyridin-2-yl)-amide
2-(2,6-Dichloro-4-morpholin-4-yl-phenyl)-1H-indole-6-carboxylic acid quinolin-2-ylamide
3-{3,5-Dimethyl-4-[6-(quinolin-2-ylcarbamoyl)-1H-indol-2-yl]-phenyl}-propionic acid methyl ester
3-{3,5-Dimethyl-4-[6-(quinolin-2-ylcarbamoyl)-1H-indol-2-yl]-phenyl}-propionic acid
3-{4-[6-(4-tert-Butylphenylcarbamoyl)-1H-indol-2-yl]-3,5-dimethylphenyl}-propionic acid
3-{3,5-Dichloro-4-[6-(quinolin-2-ylcarbamoyl)-1H-indol-2-yl]-phenyl}-propionic acid hydrochloride salt
2-(2,6-Dichloro-4-hydroxyphenyl)-1H-indole-6-carboxylic acid quinolin-2-ylamide
3-{4-[5-(3,4-Dimethylphenylcarbamoyl)-1H-benzoimidazol-2-yl]-3,5-dimethylphenyl}-propionic acid
3-{4-[6-(3,4-Dimethylphenylcarbamoyl)-1H-benzoimidazol-2-yl]-3,5-dimethylphenyl}-propionic acid methyl ester
3-{4-[6-(5,6-Dimethylpyridin-2-ylcarbamoyl)-1H-benzimidazol-2-yl]-3,5-dimethylphenyl}-propionic acid
{3,5-Dichloro-4-[6-(3,4-dimethylphenylcarbamoyl)-1H-benzoimidazol-2-yl]-phenoxy}-acetic acid methyl ester
{3,5-Dichloro-4-[6-(3,4-dimethyl-phenylcarbamoyl)-1H-benzoimidazol-2-yl]-phenoxy}-acetic acid
{3-Chloro-4-[6-(3,4-dimethylphenylcarbamoyl)-1H-benzoimidazol-2-yl]-phenoxy}-acetic acid methyl ester
{3-Chloro-4-[6-(3,4-dimethylphenylcarbamoyl)-1H-benzoimidazol-2-yl]-phenoxy}-acetic acid
{3,5-Dimethyl-4-[6-(quinolin-2-ylcarbamoyl)-1H-benzoimidazol-2-yl]-phenoxy}-acetic acid methyl ester
{3,5-Dimethyl-4-[6-(quinolin-2-ylcarbamoyl)-1H-benzoimidazol-2-yl]-phenoxy}-acetic acid
2-[4-((R)-2,2-Dimethyl-[1,3]dioxolan-4-ylmethoxy)-2,6-dimethylphenyl]-3H-benzoimidazole-5-carboxylic acid (3,4-dimethylphenyl)-amide
2-[4-((R)-2,3-Dihydroxy-propoxy)-2,6-dimethylphenyl]-3H-benzoimidazole-5-carboxylic acid (3,4-dimethylphenyl)-amide
2-[4-((S)-2,2Dimethyl-[1,3]dioxolan-4-ylmethoxy)-2,6-dimethylphenyl]-3H-benzoimidazole-5-carboxylic acid (3,4-dimethylphenyl)-amide
2-[4-((R)-2,3-Dihydroxy-propoxy)-2,6-dimethylphenyl]-3H-benzimidazole-5-carboxylic acid (3,4-dimethylphenyl)-amide
2-[4-((R)-2,2-Dimethyl-[1,3]dioxolan-4-ylmethoxy)-2,6-dimethylphenyl]-3H-benzoimidazole-5-carboxylic acid quinolin-2-ylamide
2-[4-((S)-2,3-Dihydroxypropoxy)-2,6-dimethylphenyl]-3H-benzoimidazole-5-carboxylic acid quinolin-2-ylamide
3-{4-[6-(Quinolin-2-ylcarbamoyl)-1H-benzoimidazol-2-yl]-phenyl}-propionic acid
3-{3,5-Dimethyl-4-[6-(naphthalen-2-ylcarbamoyl)-1H-benzoimidazol-2-yl]-phenyl}-propionic acid
3-{4-[6-(Isoquinolin-1-ylcarbamoyl)-1H-benzoimidazol-2-yl]-3,5-dimethylphenyl}-propionic acid
{3,5-Dichloro-4-[6-(quinolin-2-ylcarbamoyl)-1H-benzoimidazol-2-yl]-phenoxy}-acetic acid methyl ester
{3,5-Dichloro-4-[6-(quinolin-2-ylcarbamoyl)-1H-benzoimidazol-2-yl]-phenoxy}-acetic acid
2-(2,6-Dichloro-4-dimethylcarbamoylmethoxyphenyl)-3H-benzoimidazole-5-carboxylic acid quinolin-2-ylamide
{3,5-Dichloro-4-[6-(quinolin-2-ylcarbamoyl)-1H-benzoimidazol-2-yl]-phenoxymethyl}-phosphonic acid diethylester
{3,5-Dichloro-4-[6-(quinolin-2-ylcarbamoyl)-1H-benzoimidazol-2-yl]-phenoxymethyl}-phosphonic acid
3-{3,5-Dichloro-4-[6-(quinolin-2-ylcarbamoyl)-1H-benzoimidazol-2-yl]-phenyl}-propionic acid
3-{4-[6-(4-tert-Butylphenylcarbamoyl)-1H-benzimidazol-2-yl]-3,5-dimethylphenyl}-propionic acid
(E)-3-{4-[6-(4-tert-Butylphenylcarbamoyl)-1H-benzimidazol-2-yl]-3,5-dimethylphenyl}-acrylic acid
{4-[6-(4-tert-Butylphenylcarbamoyl)-1H-benzoimidazol-2-yl]-3,5-dimethylphenoxy}-acetic acid
3-{4-[6-(4-tert-Butylphenylcarbamoyl)-1H-benzoimidazol-2-yl]-3,5-dimethylphenyl}-2,2-dimethylpropionic acid
3-{3,5-Dimethyl-4-[6-(quinolin-2-ylcarbamoyl)-1H-benzoimidazol-2-yl]-phenyl}-2,2-dimethyl-propionic acid
3-{3,5-Dimethyl-4-[5-(6-trifluoromethyl-pyridin-3-ylcarbamoyl)-1H-benzoimidazol-2-yl]-phenyl}-2,2-dimethylpropionic acid
(2-{3,5-Dimethyl-4-[6-(quinolin-2-ylcarbamoyl)-1H-benzoimidazol-2-yl]-phenyl}-ethyl)-phosphonic acid
(3-{3,5-Dimethyl-4-[6-(quinolin-2-ylcarbamoyl)-1H-benzoimidazol-2-yl]-phenyl}-propyl)-phosphonic acid diethyl ester
(3-{3,5-Dimethyl-4-[6-(quinolin-2-ylcarbamoyl)-1H-benzoimidazol-2-yl]-phenyl}-propyl)-phosphonic acid
(3-{3,5-Dimethyl-4-[6-(quinolin-2-ylcarbamoyl)-1H-benzoimidazol-2-yl]-phenyl}-propyl)-phosphonic acid monoethyl ester
(3-{3,5-Dimethyl-4-[6-(6-trifluoromethyl-pyridin-3-ylcarbamoyl)-1H-benzoimidazol-2-yl]-phenyl}-propyl)-phosphonic acid
(3-{4-[6-(4-tert-Butyl-phenylcarbamoyl)-1H-benzoimidazol-2-yl]-3,5-dimethyl-phenyl}-propyl)-phosphonic acid
3-{3,5-Dichloro-4-[6-(6-trifluoromethylpyridin-3-ylcarbamoyl)-1H-benzoimidazol-2-yl]-phenyl}-propionic acid
(3,4-Dimethylphenyl)-{1-[2-(2,6-dimethylphenyl)-3H-benzoimidazol-5-yl]-2,2,2-trifluoroethyl}-amine
3-{3,5-Dimethyl-4-[6-(quinolin-2-ylcarbamoyl)-1H-benzoimidazol-2-yl]-phenyl}-propionic acid
   or any pharmaceutically acceptable salt or prodrug thereof.

### Methods of Preparation of Compounds Disclosed in PCT/US2007/081607:

In the below description of general Methods of Preparation and Synthesis;
Ar can represent D-C— wherein D and C are as hereinabove defined.
ArCHO can represent D-C—CHO, wherein D and C are as hereinabove defined.
RNH₂ can represent H₂N—(CH₂)ₙ—(CR₄R_{4′})ᵣ(CH₂)-A or A-(CH₂)ₙ—(CR₄R_{4′})ₚ (CH₂)ₘ—NH₂ wherein A, R₄, R_{4′}, p, n and m have the same definition as for L1 which are hereinabove defined.
RCOCl can represent Cl—C(O)—(CH₂)ₙ—(CR₄R_{4′})ₚ—(CH₂)ₙ-A or A-(CH₂)ₙ—(CR₄R_{4′})ₚ—(CH₂)ₘ—C(O)—Cl wherein A, R₄, R_{4′}, p, n and m have the same definition as for L1 which are hereinabove defined.
RSO₂Cl can represent Cl—SO₂—(CH₂)ₙ—(CR₄R_{4′})ₚ—(CH₂)ₘ-A or A-(CH₂)—(CR₄R_{4′})ₚ (CH₂)—SO₂—Cl wherein A, R₄R_{4′}, p, n and m have the same definition as for L1 which are hereinabove defined.
Y can represent A-L1-.
R₂ can represent a hereinabove defined heterocyclyl substituent.
≡—Ar can represent ≡—C-D wherein D and C are as hereinabove defined.

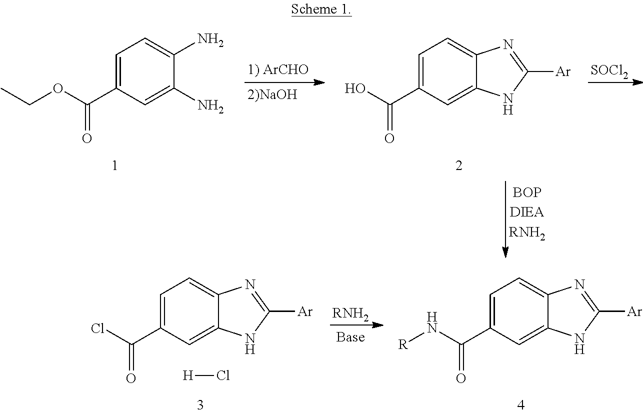

Oxidative cyclocondensation of 3,4-diamino-benzoic acid ethyl ester with substituted benzaldehyde provides the benzimidazole core. The reaction is carried out in the open air in oxidizing media, such as DMSO or nitrobenzene, preferably the former, in the presence of a catalyst such as FeCl3, Sc(OTf)₃/Cu(OTf)₂, or Yb(OTf)₃/Cu(OTf)₂. After saponification of the ethyl ester, resulting carboxylic acid is converted to acid chloride by the action of SOCl2 and ensuing amidation with a variety of alkyl, aryl, or heteroarylamine in the presence of base such as, but not limited to, DIPEA, pyridine, or Na2CO3, affords compounds 4. In addition, amidation of compound 2 by using an coupling reagent such as, but not limited to, BOP and EDCI with aryl, heteroarylamine in an inert solvent can provides compounds 4.

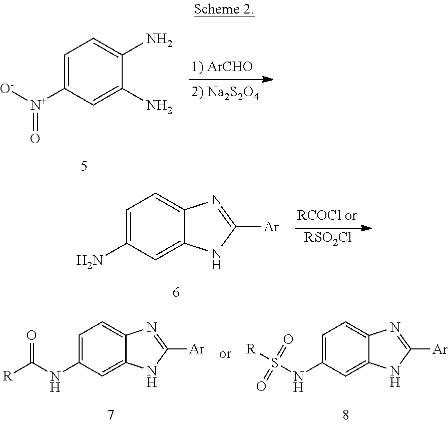

In the similar fashion, 4-nitrophenylenediamine is converted to 2-aryl-5-nitro-3H-benzoimidazole, which, upon reduction of the nitro group, give rise to aerobically unstable 5-aminobenzimidazole core. Amidation or sulfonylation of the latter provides a variety of 2-aryl-3H-benzoimidazol-5-amine carboxamides 7 and sulfonamides 8.

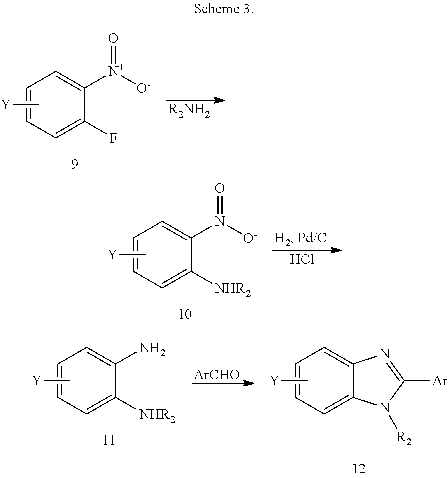

In another form of synthesis, the cyclocondensation can be carried out at the last stage, with the eventual 5-substituent pre-installed on the ring.

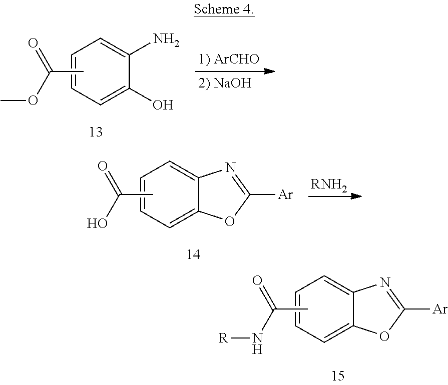

Oxidative cyclocondensation of compound 13 and subsequent hydrolysis can provide compound 14. The amidation of compound 14 by using an coupling reagent can afford compound 15.

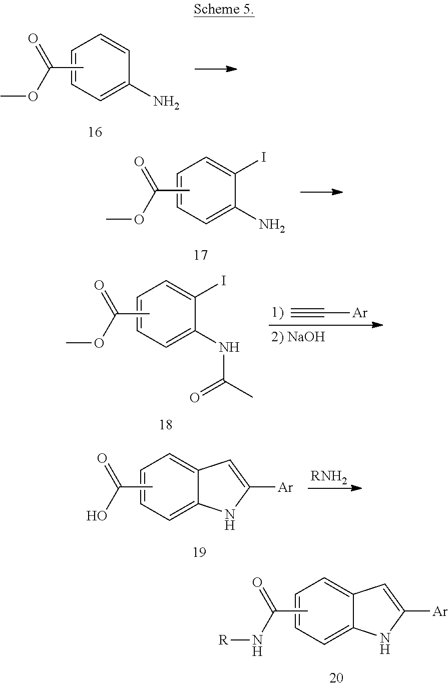

Compound 19, useful for the preparation of compound 20 can be prepared by employing the palladium-catalyzed coupling of alkynes with iodoanilines in the present of TMG (tetramethylguanidine).

HPLC Method 10: 4.6 mm×5 cm Inersil C8-3 reverse phase, 3.0 μm particle size running a gradient of 10-90% MeCN/water (5 mM ammonium formate) over a period of 2 min at a flow rate of 4 mL/min at 50° C. DAD-UV detection, 220-600 nm.

The following Examples are intended to illustrate the invention and are not to be construed as being limitations thereon. If not mentioned otherwise, all evaporations are performed under reduced pressure, preferably between about 50 mmHg and 100 mmHg. The structure of final products, intermediates and starting materials is confirmed by standard analytical methods, e.g., microanalysis, melting point (m.p.) and spectroscopic characteristics, e.g., MS, IR and NMR. Abbreviations used are those conventional in the art.

### Example 1-1

### [2-(2-Chloro-phenyl)-3H-benzoimidazol-5-yl]-carbamic acid ethyl ester

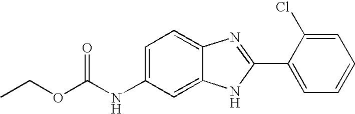

To a solution of 4-nitro-benzene-1,3-diamine (1.6 g) in acetonitrile (20 mL) was added NaHCO₃ (1 g) and ethyl chloroformate (1.0 mL). The mixture was heated at 75° C. with stirring overnight. Cooled to ambient temperature, the mixture was diluted by water and extracted twice with ethyl acetate. Combined organics were dried over Na₂SO₄, filtered, and concentrated to give crude yellow solid. Trituration from dichloromethane afforded (3-amino-4-nitro-phenyl)-carbamic acid ethyl ester: ¹H NMR (400 MHz, CD₃OD) 7.9 (d, 1H), 7.3 (s, 1H), 6.5 (d, 1H), 4.2 (q, 2H), 1.3 (t, 3H).

A solution of (3-amino-4-nitro-phenyl)-carbamic acid ethyl ester (1.2 g) in ethyl acetate (40 mL) was placed in a Parr shaker bottle and 10% Pd/C (200 mg) was added. The mixture was hydrogenated at 50 psi H₂ for 20 h. The mixture was filtered on Celite and the filtrated was treated with 4 M HCl in dioxane (5 mL). Resulting precipitate was filtered and washed with ethyl acetate before it was vacuum-dried to give (3,4-diamino-phenyl)-carbamic acid ethyl ester di-hydrochloride salt as a purple solid: ¹H NMR (400 MHz, DMSO-d6) 9.5 (s, 1H), 7.1 (s, 1H), 7.0 (d, 1H), 6.8 (d, 1H), 4.1 (q, 2H), 1.3 (t, 3H).

A freshly prepared solution of (3,4-diamino-phenyl)-carbamic acid ethyl ester in DMSO (0.2 M, 0.10 mL) was placed in a vial. To it was added 2-chloro-benzaldehyde (0.2 M in toluene, 0.12 mL), followed by FeCl₃ (0.02 M in THF, 0.050 mL). The mixture was stirred in open air at ambient temperature overnight. The mixture was then diluted by MeOH and the whole was loaded onto a solid phase extraction (SPE) cartridge that contained strong cation exchange (SCX) (Ig media in 6 mL cartridge, United Chemical Technology). Wash-to-waste (5 mL MeOH) was followed by elute-to-collect (5 mL 20:2:1 ethyl acetate-MeOH-Et3N) and, after evaporation of volatiles, the crude was further purified by silica gel column chromatography to give [2-(2-chloro-phenyl)-3H-benzoimidazol-5-yl]-carbamic acid ethyl ester: MS (m/z) 315.97 (M+1).

### Example 2-1

### 2-(4-Chloro-phenyl)-3-(2-hydroxyethyl)-3H-benzoimidazole-5-carboxylic acid (3,4-dimethyl-phenyl)-amide

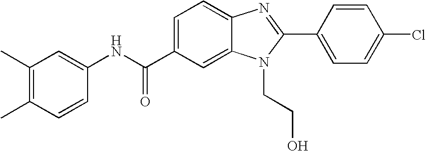

A solution of 3-fluoro-4-nitrobenzoic acid (2 g), 3,4-dimethylaniline (1.25 g), BOP (5.45 g) and DIEA (2.69 mL) in DMF (20 mL) was stirred at room temperature for 20 h. Then 1N NaOH aqueous solution was added (pH ˜12). The aqueous layer was extracted with EtOAc. The organic extracts were washed with brine, dried over Na₂SO₄, filtered and concentrated under reduced pressure. The crude product was washed with CH₂Cl₂/MeOH mixture, and the solid was filtered to give N-(3,4-dimethyl-phenyl)-3-fluoro-4-nitrobenzamide as a yellow solid: MS (m/z) 289 (M+1); ¹H NMR (DMSO-d6, 400 MHz) δ10.39 (s, 1H), 8.30 (t, 1H), 8.10 (d, 1H), 7.97 (d, 1H), 7.53 (s, 1H), 7.49 (d, 1H), 7.13 (d, 1H), 2.23 (s, 3H), 2.20 (s, 3H).

A mixture of N-(3,4-dimethyl-phenyl)-3-fluoro-4-nitrobenzamide (65 mg), 2-hydroxy-1-ethylamine (0.225 mL of 2M THF solution) in DMF (1 mL) was heated at 120° C. under microwave radiation for 5 min. Then saturated NaHCO₃ aqueous solution was added. The aqueous layer was extracted with EtOAc. The organic extracts were washed with brine, dried over Na₂SO₄, filtered and concentrated under reduced pressure to give N-(3,4-dimethyl-phenyl)-3-(2-hydroxy-ethylamino)-4-nitrobenzamide as a yellow oil: MS (m/z) 300 (M+1); ¹H NMR (CDCl₃, 400 MHz) δ8.24 (d, 1H), 8.06 (s, 1H), 7.78 (s, 1H), 7.42 (d, 2H), 7.35 (d, 1H), 7.13 (d, 1H), 6.95 (d, 1H), 3.09 (d, 3H), 2.28 (s, 3H), 2.25 (s, 3H), 1.58 (s, 1H).

To a solution of N-(3,4-dimethyl-phenyl)-3-(2-hydroxyethylamino)-4-nitro-benzamide (70 mg) in MeOH (20 mL) was added 10% Pd/C (10 mg). The reaction mixture was stirred under H₂ balloon at room temperature for 16 h. The reaction mixture was filtered through celite, washed with MeOH. The filtrate was concentrated under reduced pressure to give 4-amino-N-(3,4-dimethyl-phenyl)-3-(2-hydroxy-ethylamino)-benzamide as a grey solid; MS (m/z) 300 (M+1); ¹H NMR (DMSO-d6, 400 MHz) δ9.54 (s, 1H), 7.51 (s, 1H), 7.45 (d, 1H), 7.18 (d, 1H), 7.04 (d, 2H), 6.57 (d, 1H), 5.12 (s, 2H), 4.69 (t, 1H), 4.50 (t, 1H), 3.65 (q, 2H), 3.19 (q, 2H), 2.20 (s, 3H), 2.17 (s, 3H).

A mixture of 4-amino-N-(3,4-dimethyl-phenyl)-3-(2-hydroxy-ethylamino)-benzamide (0.1 mL in 0.2 M DMSO solution), 4-chlorobenzaldehyde (0.1 mL in 0.2 M toluene solution) and FeCl₃ (0.05 mL in 0.02 M THF solution) was stirred in open air at ambient temperature overnight. The mixture was then diluted by MeOH and the whole was loaded onto a solid phase extraction (SPE) cartridge that contained strong cation exchange (SCX) (1 g media in 6 mL cartridge, United Chemical Technology). Wash-to-waste (5 mL MeOH) was followed by elute-to-collect (5 mL 20:2:1 ethyl acetate-MeOH-Et3N) and, after evaporation of volatiles, the crude was further purified by silica gel column chromatography to give 2-(4-chlorophenyl)-3-(2-hydroxy-ethyl)-3H-benzoimidazole-5-carboxylicacid (3,4-dimethylphenyl)-amide: MS (m/z) 420 (M+1).

### Example 3-1

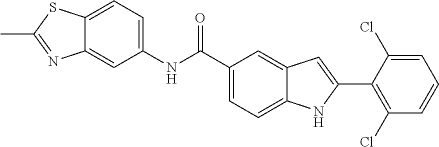

To a solution of 4-amino-benzoic acid methyl ester (Ig) in 10 mL of acetic acid and 10 mL of CH₂Cl₂ was added benzyltrimethylammonium dichloroiodate (2.763 g) at ambient temperature. The reaction mixture was heated at 55° C. for 1.5 h. The reaction mixture was concentrated to give the crude 4-amino-3-iodo-benzoic acid methyl ester: MS (m/z) 278.0 (M+1).

To a solution of the crude 4-amino-3-iodo-benzoic acid methyl ester in 20 mL of acetic acid was added acetic anhydride (1.25 mL). The reaction was heated at 60° C. for 1 h. The reaction mixture was quenched with water, extracted with ethyl acetate, dried over Na₂SO₄, and concentrated in vacuo. Purification by flash chromatography (SiO₂, 1:3 EtOAc/Hexane) afforded 4-acetylamino-3-iodo-benzoic acid methyl ester: MS (m/z) 320.0 (M+1).

To a solution of 4-acetylamino-3-iodo-benzoic acid methyl ester (0.17 g) in TMG (1.5 mL) and dioxane (1.5 mL) were added 1,3-dichloro-2-ethynyl-benzene (0.1 g), 10 mol % of Pd(PPh₃)₂Cl₂, and 10 mol % of CuI. The reaction mixture was heated to 100° C. overnight. The solvent were evaporated and the resulting mixture was purified by column chromatography (SiO₂, 2:3, EtOAc/Hexane) to give 2-(2,6-dichloro-phenyl)-1H-indole-5-carboxylic acid methyl ester: MS (m/z) 320.0 (M+1).

A solution of 2-(2,6-dichloro-phenyl)-1H-indole-5-carboxylic acid methyl ester in 2N LiOH aqueous solution (1 mL) and THF (1 mL) was stirred at room temperature for 16 h. The mixture was washed with Et₂O. The aqueous layer was acidified with 1N HCl aqueous solution, extracted with Et₂O. The organic extracts were dried over Na₂SO₄, filtered and concentrated under reduced pressure to give 2-(2,6-dichloro-phenyl)-1H-indole-5-carboxylic acid as a white solid: MS (m/z) 306.1 (M+1).

To a solution of 2-(2,6-dichloro-phenyl)-1H-indole-5-carboxylic acid (8.8 mg) in anhydrous DMF (0.5 mL) was added BOP (17.4 mg), diisopropylethylamine (0.014 mL), and 3,4-dimethylaniline (5.0 mg). The reaction mixture was stirred at 85° C. overnight, quenched with 1N NaOH aqueous solution at 0° C., extracted with EtOAc. The extracts were dried over Na₂SO₄, and concentrated in vacuo. The crude reaction mixture was purified by silica SPE eluting with EtOAc/Hexane to give 2-(2,6-dichloro-phenyl)-1H-indole-5-carboxylic acid (2-methyl-benzothiazol-5-yl)-amide: MS (m/z) 452.1 (M+1).

### Example 4-1

### 2-(2,6-dichloro-phenyl)-benzooxazole-6-carboxylic acid (2-o-tolyl-ethyl)-amide

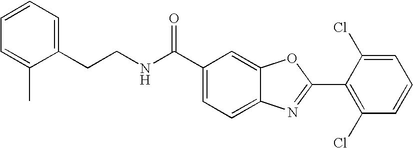

To a solution of 4-amino-3-hydroxy-benzoic acid methyl ester (2.0 g) in MeOH (100 mL) was added 2,6-dichlorobenzaldehyde (2.1 g). The reaction mixture was heated at 45° C. overnight, and then concentrated in vacuo. The residue was dissolved in THF (10 mL) and dichloromethane (90 mL). To the mixture solution was added 2,3-dichloro-5,6-dicyano-1,4-benzoquinone (2.72 g). The reaction mixture was stirred for 1 h, diluted with NaHCO₃ aqueous solution, extracted with EtOAc, and washed with brine. The extracts were dried over Na₂SO₄ and concentrated in vacuo. Purification by column chromatography (SiO₂, 2:3 EtOAc/Hexane) gave 2-(2,6-dichlorophenyl)-benzooxazole-6-carboxylic acid methyl ester: MS (m/z) 321.9 (M+1).

To a solution of 2-(2,6-dichlorophenyl)-benzooxazole-6-carboxylic acid methyl ester (1.6 g) in EtOH (13 mL) was added 4N NaOH aqueous solution (6.2 mL). The reaction was stirred at 80° C. for 0.5 h. The reaction mixture was concentrated, diluted with water, and treated with Et₂O. The aqueous layer was acidified with 1N HCl aqueous solution. The precipitate was filtered, washed with water, and dried in vacuo to give 2-(2,6-dichloro-phenyl)-benzooxazole-6-carboxylic acid as a white solid: MS (m/z) 308.0 (M+1).

To a solution of 2-(2,6-dichloro-phenyl)-benzooxazole-6-carboxylic acid (8.9 mg) in anhydrous DMF (0.5 mL) was added BOP (17.4 mg), diisopropylethylamine (0.014 mL), and 2-o-tolyl-ethylamine (5.5 mg). The reaction mixture was stirred at 85° C. overnight, quenched with 1N NaOH aqueous solution at 0° C., extracted with EtOAc. The extracts were dried over Na₂SO₄ and concentrated in vacuo. The crude reaction mixture was purified by silica SPE eluting with EtOAc/Hexane to give 2-(2,6-dichlorophenyl)-benzooxazole-6-carboxylic acid (2-o-tolyl-ethyl)-amide: MS (m/z) 425.1 (M+1).

### Example 5-1

### {4-[5-(3,4-Dimethyl-phenylcarbamoyl)-1H-benzoimidazol-2-yl]-3,5-dimethyl-phenylamino}-acetic acid methyl ester

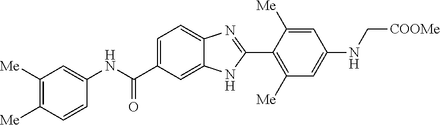

To a solution of 3,5-dimethylphenylamine (7.26 g) in 200 mL of CH₂Cl₂, cooled in an ice bath, was added trifluoroacetic anhydride (12.5 mL) slowly. After the addition, the solution was stirred at room temperature for 15 min then Br₂ (2.93 mL) was added slowly while maintaining room temperature with a water bath. The solution was stirred at room temperature for 3.5 h then was quenched with 10% Na₂S₂O₃. The aqueous layer was extracted with CH₂Cl₂ The combined organic layers were dried and filtered. Removal of the solvent under reduced pressure gave an orange solid which was purified by recrystallization (1:1 hexanes/ethyl ether) to give N-(4-bromo-3,5-dimethylphenyl)-2,2,2-trifluoroacetamide as a white solid. MS (ESI) m/z 297 (M+H).

To a solution of N-(4-bromo-3,5-dimethylphenyl)-2,2,2-trifluoroacetamide (592 mg) in THF (10 mL) at −78° C. was added MeL1/LiBr (1.5M in Et₂₀, 1.87 mL) slowly. After 5 min, sec-BuLi (1.4M. in cyclohexane, 2.0 mL) was added slowly to the solution at −78° C. After 5 min DMF (0.31 mL) was added to the solution drowise at −78° C. then the mixture was warmed to room temperature. After 30 min the reaction mixture was partioned between water and CH₂Cl₂. The aqueous layer was extracted with CH₂Cl₂ and the combined organic layers were dried and filtered. Removal of the solvent under reduced pressure to gave a yellow solid which was purified by flash chromatography using hexanes/ethyl acetate (5:1) as eluent which gave 2,2,2-trifluoro-N-(4-formyl-3,5-dimethyl-phenyl)-acetamide as a light yellow solid. MS (ESI) m/z 246 (M+H).

To a solution of 2,2,2-trifluoro-N-(4-formyl-3,5-dimethylphenyl)-acetamide (1.0 g) in MeOH (20 mL) was added 1N NaOH (16.3 mL). After 4 hrs at room temperature, the suspension was filtered and washed with water. The solid was dried under reduced pressure to give 4-amino-2,6-dimethylbenzaldehyde as a light yellow solid. MS (ESI) m/z 150 (M+H).

To a solution of 4-amino-2,6-dimethylbenzaldehyde (600 mg) and bromoacetic acid methyl ester (0.4 mL) in DMF (20 mL) was added K₂CO₃ (1.4 g) and the suspension was heated at 80° C. After 1 h, additional bromoacetic acid methyl ester (0.4 mL) was added and this process was continued until the starting material was consumed (check by LC/MS). The reaction mixture was partioned between water and EtOAc. The aqueous layer was extracted with EtOAc. The combined organic layers were dried, filtered and concentrated and the residue was purified by flash chromatography using hexanes/ethyl:acetate (2:1) to give (4-formyl-3,5-dimethylphenylamino)-acetic acid methyl ester as a yellow solid. MS (ESI) m/z 222 (M+H).

To a solution of 4-amino-3-nitrobenzoic acid (1.82 g) in in DMF (20 mL) was added HOBT (1.49 g) and EDCI (2.1 g). After stirring at room temperature for 10 min, 3,4-dimethylphenylamine (1.2 g) and DIPEA (5.3 mL) were added. The solution was stirred at room temperature for 18 h then the mixture was partioned between water and EtOAc The aqueous layer was extracted with EtOAc. The combined organic layers were washed with water, brine, dried, filtered and concentrated. The residue was purified by recrystallization from EtOAc and gave 4-amino-N-(3,4-dimethylphenyl)-3-nitrobenzamide as a yellow solid. MS (ESI) m/z 286 (M+H).

A solution of 4-amino-N-(3,4-dimethylphenyl)-3-nitro-benzamide (2.0 g) in EtOH (40 mL) was hydrogenated at 1 atm over PtO₂ (200 mg, 10% w) for 18 hrs. The catalyst was filtered through Celite and the solvent was removed under reduced pressure to afford 3,4-diamino-N-(3,4-dimethylphenyl)-benzamide as a yellow solid. MS (ESI) m/z 256 (M+H).

To a solution of (4-formyl-3,5-dimethylphenylamino)-acetic acid methyl ester (800 mg) and 3,4-diamino-N-(3,4-dimethylphenyl)-benzamide (694 mg) in DMSO (15 mL) was added Yb(OTf)₃ (390 mg) and Cu(OTf)₂ (228 mg); The solution was stirred at room temperature for 18 h then the reaction mixture was partioned between water and EtOAc. The aqueous layer was extracted with EtOAc. The combined organic layers were dried, filtered and concentrated. The residue was purified by flash chromatography (amino-column) using hexanes/ethyl acetate (1:4) to give {4-[5-(3,4-dimethylphenylcarbamoyl)-1H-benzoimidazol-2-yl]-3,5-dimethylphenylamino}-acetic acid methyl ester as a light yellow solid. MS (ESI) m/z 457 (M+H); ¹H NMR (MeOD, 400 MHz) δ 8.21 (s, 1H), 7.85 (dd, 1H), 7.67 (s, 1H), 7.47 (d, 1H), 7.42 (dd, 1H), 7.12 (d, 1H), 6.42 (s, 2H), 3.97 (s, 2H), 3.75 (s, 3H), 2.29 (s, 3H), 2.26 (s, 3H), 2.09 (s, 6H).

### Example 6-1

### 3-{4-[5-(3,4-Dimethylphenylcarbamoyl)-1H-benzoimidazol-2-yl]-3,5-dimethylphenyl}-propionic acid tert-butyl ester

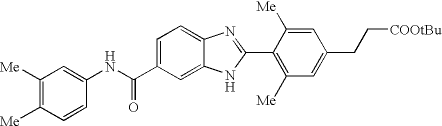

A solution of NaNO₂ (102 mg) in water (1 mL) was added to an ice cold mixture of 4-amino-2,6-dimethylbenzaldehyde (Example 5-1, step 3) (220 mg) and 480% HBF₄ (0.5 mL). After 30 min at 0° C., tert-butyl acrylate (0.43 mL) and Pd(OAc)₂ (10 mg) were added and the mixture was heated to 80° C. (or in a water bath) for 30 min. The suspension was filtered through Celite, washed with CH₂Cl₂ and the filtrate extracted with CH₂Cl₂. The combined organic layers were dried, filtered and concentrated. The residue was purified by flash chromatography using hexanes/ethyl acetate (7:1) and gave 3-(4-formyl-3,5-dimethylphenyl)-acrylic acid tert-butyl ester.

A solution of 3-(4-formyl-3,5-dimethylphenyl)-acrylic acid tert-butyl ester (210 mg) in CH₂Cl₂ (8 mL) was hydrogenated at 1 atm over 10% Pd/C (21 mg) for 4 h. The catalyst was filtered and the filtrate concentrated to give 3-(4-formyl-3,5-dimethylphenyl)-propionic acid tert-butyl ester as a yellow solid. MS (ESI) m/z 286 (M+H).

To a solution of 3-(4-formyl-3,5-dimethylphenyl)-propionic acid tert-butyl ester (200 mg) and 3,4-diamino-N-(3,4-dimethylphenyl)-benzamide (Example 5-1, step 6) (194 mg) in DMSO (10 mL) was added Yb(OTf)₃ (93 mg). The solution was stirred at room temperature for 18 h then the mixture was partioned between water and EtOAc. The aqueous layer was extracted with EtOAc. The combined organic layers were dried, filtered and concentrated. The residue was purified by flash chromatography (amino-column) using hexanes/ethyl acetate (1:4) and gave 3-{4-[5-(3,4-dimethylphenylcarbamoyl)-1H-benzoimidazol-2-yl]-3,5-dimethylphenyl}-propionic acid tert-butyl ester as a red solid. MS (ESI) m/z 498 (M+H). ¹H NMR (Acetone-d₆, 400 MHz) δ 11.73 (s, 1H), 9.39 (s, 1H), 8.30 (s, 1H), 7.94 (t, 1H), 7.69 (m, 3H), 7.12 (s, 1H), 7.07 (s, 2H), 2.90 (t, 2H), 2.58 (t, 2H), 2.28 (s, 3H), 2.25 (s, 3H), 2.16 (s, 6H), 1.45 (s, 9H).

### Example 7-1

### 2-(2,6-Dimethylphenyl)-1,3-dioxo-2,3-dihydro-1H-isoindole-5-carboxylic acid (3,4-dimethylphenyl)-amide

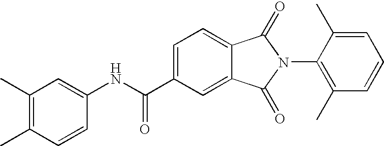

To a solution of 2,6-dimethylaniline (1.21 g) in DMF (15 mL) was added 1,3-dioxo-1,3-dihydroisobenzofuran-5-carboxylic acid (1.92 g) and the mixture was stirred at room temperature for 18 h. The mixture was poured into water and the resulting precipitate was filtered, washed with water and dried under reduced pressure. The solid was suspended in HOAc (25 mL) and the mixture was heated at 110° C. for 4 h. The resulting solution was cooled and the solvent was removed under reduced pressure to give 2-(2,6-dimethylphenyl)-1,3-dioxo-2,3-dihydro-1H-isoindole-5-carboxylic acid as an off-white solid, mp=210-213° C.; MS (m/z) 294 (M−1).

To a mixture of 2-(2,6-dimethylphenyl)-1,3-dioxo-2,3-dihydro-1H-iso indole-5-carboxylic acid (130 μg), EDCI (85 mg) and HOBt (60 mg) in DMF (3 mL) was added 3,4-dimethylaniline (53 mg) and the mixture was stirred at room temperature for 24 h. The mixture was poured into water and was extracted with EtOAc. The organic phase was washed with water (3×) and dried over sodium sulfate. The solvent was removed under reduced pressure and the residue purified by flash chromatography using CH₂Cl₂ as eluent to give 2-(2,6-dimethylphenyl)-1,3-dioxo-2,3-dihydro-1H-isoindole-5-carboxylic acid (3,4-dimethylphenyl)-amide as a pale-yellow solid, mp=223-225° C.; MS (m/z) 397 (M−1); ¹H NMR (CDCl₃, 400 MHz) δ 8.38 (s, 1H), 8.09 (d, J=8.34 Hz, 1H), 7.81 (s, 1H), 7.45 (s, 1H), 7.37-7.15 (m, 5H), 2.30 (s, 3H), 2.27 (s, 3H), 1.54 (s, 6H).

### Example 8-1

### (3,4-Dimethylphenyl)-{1-[2-(2,6-dimethylphenyl)-3H-benzoimidazol-5-yl]-2,2,2-trifluoroethyl}-amine

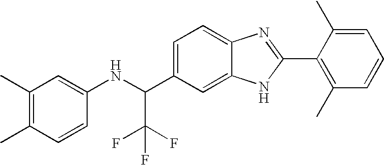

To a solution of 2-(2,6-dimethylphenyl)-3H-benzoimidazole-5-carboxylic acid methyl ester (930 mg) (Example 1-255, step 1) in THF (10 mL) was added 16.6 mL of a 1M solution of LiAlH₄ in ether dropwise. The mixture was stirred at ambient temperature for 18 h then approximately 4 mL of saturated sodium sulfate solution was added dropwide. Ethyl acetate was added to the mixture and the solvent decanted from any insoluble material. The organic solution was dried over sodium sulfate and the solvent was removed under reduced pressure to give, [2-(2,6-dimethylphenyl)-3H-benzoimidazol-5-yl]-methanol as a foam.

A mixture of [2-(2,6-dimethylphenyl)-3H-benzoimidazol-5-yl]-methanol (750 mg) and MnO₂ (5 g) in THF (10 mL) was stirred at ambient temperature for 4 h. The mixture was filtered through Celite and the filtrate evaporated to give 2-(2,6-dimethyl-phenyl)-3H-benzoimidazole-5-carbaldehyde as an oil. MS (ESI) m/z 251 (M+H).

To a solution of 2-(2,6-dimethylphenyl)-3H-benzoimidazole-5-carbaldehyde as an oil (590 mg) and Boc₂O (515 mg) in THF (5 mL) was added DMAP (25 mg) and the mixture was stirred at ambient temperature for 18 h. The solvent was removed under reduced pressure and the residue was purified by flash chromatography using 10% EtOAc/methylene chloride as eluent to give 2-(2,6-dimethylphenyl)-6-formyl-benzoimidazole-1-carboxylic acid tert-butyl ester as an oil.

To 2-(2,6-dimethylphenyl)-6-formyl-benzoimidazole-1-carboxylic acid tert-butyl ester (565 mg) was added 16 mL of a 0.5 M solution of trifluoromethyltrimethylsilane in THF. When a solution formed the mixture was cooled to −30° C. then a solution of TBAF (1.76 mL of a 1.0M solution in THF) was added dropwise. The mixture was stirred at −30° C. for 45 min then was allowed to warm to 5° C. The mixture was extracted with EtOAc (2×) and the combined organic layers were dried over sodium sulfate. The solvent was removed under reduced pressure and the resulting foam was purified by flash chromatography using 10% EtOAc/methylene chloride as eluent to give 2-(2,6-dimethyl-phenyl)-6-(2,2,2-trifluoro-1-hydroxyethyl)-benzoimidazole-1-carboxylic acid tert-butyl ester as a waxy solid. MS (ESI) m/z 421 (M+H).

In addition to the above material, the de-Boc analog 1-[2-(2,6-dimethylphenyl)-3H-benzoimidazol-5-yl]-2,2,2-trifluoroethanol was also isolated. MS (ESI) m/z 321.1 (M+H), retention time=3.80 min, Method 10.

To a solution of 2-(2,6-dimethylphenyl)-6-(2,2,2-trifluoro-1-hydroxyethyl)-benzoimidazole-1-carboxylic acid tert-butyl ester (475 mg) in methylene chloride (15 mL) was added Dess-Martin reagent (527 mg) and the mixture was stirred at ambient temperature for 18 h. The mixture was washed with water and sodium bicarbonate solution then was dried over sodium sulfate. The solvent was removed under reduced pressure to give 2-(2,6-dimethylphenyl)-6-(2,2,2-trifluoroacetyl)-benzoimidazole-1-carboxylic acid tert-butyl ester as a gummy solid. The material was used directly in the next reaction.

To a solution of 2-(2,6-dimethylphenyl)-6-(2,2,2-trifluoroacetyl)-benzoimidazole-1-carboxylic acid tert-butyl ester (617 mg) and 3,4-dimethylaniline (118 mg) in toluene (10 mL) was added 4 Å molecular sieves and pTos-OH (50 mg) then the mixture was stirred at 120° C. for 18 h. The sieves were filtered and the filtrate evaporated under reduced pressure to give 1-[2-(2,6-dimethylphenyl)-3H-benzoimidazol-5-yl]-2,2,2-trifluoro-ethanone as an oil.

To a solution of 1-[2-(2,6-dimethylphenyl)-3H-benzoimidazol-5-yl]-2,2,2-trifluoro-ethanone (710 mg), 3,4-dimethylaniline (150 mg) and diisopropylethylamine (150 mg) in methylene chloride (15 mL) was added dropwise 1.1 mL of a 1.0M solution of TiCl₄ in methylene chloride. The mixture was stirred at ambient temperature for 10 min then was washed with NaHCO₃ solution. The organic phase was dried over sodium sulfate and the solvent was removed under reduced pressure. The residue was purified by flash chromatography using 10% EtOAc/methylene chloride as eluent to give (3,4-dimethyl-phenyl)-[1-[2-(2,6-dimethylphenyl)-3H-benzoimidazol-5-yl]-2,2,2-trifluoroeth-(Z)-ylidene]-amine as an oil. This was used directly in the next reaction.

To a solution of (3,4-dimethyl-phenyl)-[1-[2-(2,6-dimethylphenyl)-3H-benzoimidazol-5-yl]-2,2,2-trifluoroeth-(Z)-ylidene]-amine in MeOH (3 mL) was added sodium borohydride. (25 mg) and the mixture was stirred at ambient temperature for 1 h. The MeOH was removed under reduced pressure and EtOAc was added to the residue. The mixture was washed with NaHCO₃ solution and the organic layer was dried over sodium sulfate. The solvent was removed under reduced pressure and the residue was purified by flash chromatography using 10% EtOAc/methylene chloride as eluent to give (3,4-Dimethylphenyl)-{1-[2-(2,6-dimethylphenyl)-3H-benzoimidazol-5-yl]-2,2,2-trifluoroethyl}-amine as a white solid. MS (ESI) m/z 424.0 (M+H), retention time=1.63 min, Method 10.

In starting compounds and intermediates which are converted to the compounds of the present invention in a manner described herein, functional groups present, such as amino, thiol, carboxyl and hydroxyl groups, are optionally protected by conventional protecting groups that are common in preparative organic chemistry. Protected amino, thiol, carboxyl and hydroxyl groups are those that can be converted under mild conditions into free amino thiol, carboxyl and hydroxyl groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxyl group, amino group, etc.), the structure and stability of the molecule of which the substituent is a part and the reaction conditions.

Well-known protecting groups that meet these conditions and their introduction and removal are described, e.g., in McOmie, “Protective Groups in Organic Chemistry”, Plenum Press, London, N.Y. (1973); and Greene and Wuts, “Protective Groups in Organic Synthesis”, John Wiley and Sons, Inc., NY (1999).

The above-mentioned reactions are carried out according to standard methods, in the presence or absence of diluent, preferably, such as are inert to the reagents and are solvents thereof, of catalysts, condensing or said other agents, respectively and/or inert atmospheres, at low temperatures, RT or elevated temperatures, preferably at or near the boiling point of the solvents used, and at atmospheric or super-atmospheric pressure. The preferred solvents, catalysts and reaction conditions are set forth in the illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

Depending on the choice of starting materials and methods, the new compounds may be in the form of one of the possible isomers or mixtures thereof, for example, as substantially pure geometric (cis or trans) isomers, diastereomers, optical isomers (antipodes), racemates or mixtures thereof. The aforesaid possible isomers or mixtures thereof are within the purview of this invention.

Any resulting mixtures of isomers can be separated on the basis of the physicochemical differences of the constituents, into the pure geometric or optical isomers, diastereomers, racemates, for example, by chromatography and/or fractional crystallization.

Finally, compounds of the invention are either obtained in the free form, or in a salt form thereof, preferably, in a pharmaceutically acceptable salt form thereof, or as a prodrug derivative thereof.

Compounds of the instant invention which contain acidic groups may be converted into salts with pharmaceutically acceptable bases. Such salts include alkali metal salts, like sodium, lithium and potassium salts; alkaline earth metal salts, like calcium and magnesium salts; ammonium salts with organic bases, e.g., trimethylamine salts, diethylamine salts, tris(hydroxymethyl)methylamine salts, dicyclohexylamine salts and N-methyl-D-glucamine salts; salts with amino acids like arginine, lysine and the like. Salts may be formed using conventional methods, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

Compounds of the invention, in general, may be converted into acid addition salts, especially pharmaceutically acceptable salts. These are formed, e.g., with inorganic acids, such as mineral acids, e.g., sulfuric acid, phosphoric or hydrohalic acid, or with organic carboxylic acids, such as (C₁-C₄)-alkanecarboxylic acids which, e.g., are unsubstituted or substituted by halogen, e.g., acetic acid, such as saturated or unsaturated dicarboxylic acids, e.g., oxalic, succinic, maleic or fumaric acid, such as hydroxycarboxylic acids, e.g., glycolic, lactic, malic, tartaric or citric acid, such as amino acids, e.g., aspartic or glutamic acid, or with organic sulfonic acids, such as (C₁-C₄)-alkylsulfonic acids, e.g., methanesulfonic acid; or arylsulfonic acids which are unsubstituted or substituted (for example by halogen). Preferred are salts formed with hydrochloric acid, maleic acid and methanesulfonic acid.

Prodrug derivatives of any compound of the invention are derivatives of said compounds which following administration release the parent compound in vivo via some chemical or physiological process, e.g., a prodrug on being brought to the physiological pH or through enzyme action is converted to the parent compound. Exemplary prodrug derivatives are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art.

In view of the close relationship between the free compounds, the prodrug derivatives and the compounds in the form of their salts, whenever a compound is referred to in this context, a prodrug derivative and a corresponding salt is also intended, provided such is possible or appropriate under the circumstances.

The compounds, including their salts, can also be obtained in the form of their hydrates, or include other solvents used for their crystallization.

The present invention further provides pharmaceutical compositions comprising a therapeutically effective amount of a pharmacologically active DGAT1 inhibitor compound of the instant invention, alone or in combination with one or more pharmaceutically acceptable carriers.

The pharmaceutical compositions according to the invention are those suitable for enteral, such as oral or rectal; transdermal and parenteral administration to mammals, including man, for the treatment of myocardial ischemia mediated by DGAT1 activity.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Accordingly, the present invention provides pharmaceutical compositions as described above for the treatment of myocardial ischemia mediated by DGAT1 activity.

The pharmaceutical compositions may contain a therapeutically effective amount of a compound of the invention as defined above, either alone or in a combination with another therapeutic agent, e.g., each at an effective therapeutic dose as reported in the art.

Such therapeutic agents include:

a) antidiabetic agents, such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as vildagliptin;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) anti-obesity agents such as orlistat or rimonabant; and d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as ditekiren, zankiren, terlakiren, aliskiren, RO 66-1132 and RO-66-1168; □-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.
e) agonists of peroxisome proliferator-activator receptors, such as fenofibrate, pioglitazone, rosiglitazone, tesaglitazar, BMS-298585, L-796449, the compounds specifically described in the patent application WO 2004/103995 i.e. compounds of examples 1 to 35 or compounds specifically listed in claim 21, or the compounds specifically described in the patent application WO 03/043985 i.e. compounds of examples 1 to 7 or compounds specifically listed in claim 19 and especially (R)-1-{4-[5-methyl-2-(4-trifluoromethyl-phenyl)-oxazol-4-ylmethoxy]-benzenesulfonyl}-2,3-dihydro-1H-indole-2-carboxylic or a salt thereof.

In each case in particular in the compound claims and the final products of the working examples, the subject matter of the final products, the pharmaceutical preparations and the claims are hereby incorporated into the present application by reference to these publications and patent applications.

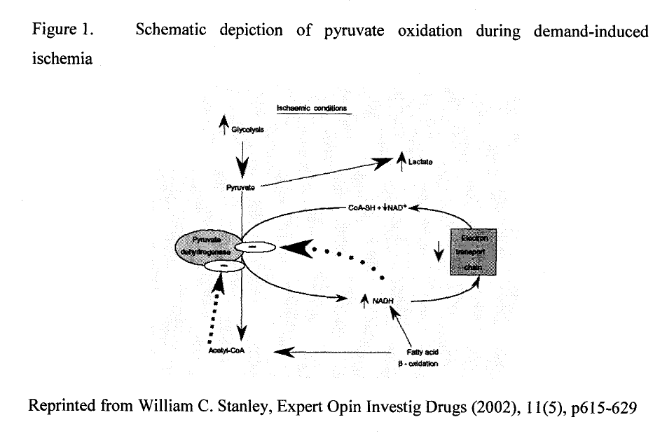

Other specific anti-diabetic compounds are described by Patel Mona in Expert Opin Investig Drugs, 2003, 12(4), 623-633, in the FIGS. 1 to 7, which are herein incorporated by reference. A DGAT1 inhibitor compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention in combination with a therapeutically effective amount of another therapeutic agent, preferably selected from anti-diabetics, hypolipidemic agents, anti-obesity agents or anti-hypertensive agents, most preferably from antidiabetics or hypolipidemic agents as described above.

The present invention further relates to pharmaceutical compositions as described above for use as a medicament.

The present invention further relates to use of pharmaceutical compositions or combinations as described above for the preparation of a medicament for the treatment of myocardial ischemia mediated by DGAT1 activity.

Thus, the present invention also relates to a pharmaceutical composition for use in myocardial ischemia mediated by DGAT1 activity comprising a DGAT1 inhibitor compound, or a pharmaceutically acceptable salt thereof, in association with a pharmaceutically acceptable diluent or carrier therefore.

The present invention further provides a method for the prevention and/or treatment of myocardial ischemia mediated by DGAT1 activity, which comprises administering a therapeutically effective amount of a DGAT inhibitor compound.

A unit dosage for a mammal of about 50-70 kg may contain between about 1 mg and 1000 mg, advantageously between about 5-500 mg of the active ingredient. The therapeutically effective dosage of active compound is dependent on the species of warm-blooded animal (mammal), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound as defined in the claims and described above, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents and anti-hypertensive agents, or a pharmaceutically acceptable salt thereof. The kit may comprise instructions for its administration.

Similarly, the present invention provides a kit of parts comprising: (i) a pharmaceutical composition of the invention; and (ii) a pharmaceutical composition comprising a compound selected from an anti-diabetic, a hypolipidemic agent, an anti-obesity agent and an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound as defined in the claims and described above, or a pharmaceutically acceptable salt thereof, and a second drug substance, said second drug substance being an anti-diabetic, a hypolipidemic agent, an anti-obesity agent or an anti-hypertensive agent, e.g., as indicated above.

Preferably, a pharmaceutical composition of the invention is administered to a human in need thereof.

Finally, the present invention provides a method or use which comprises administering a DGATl inhibitor compound as defined and described above in combination with a therapeutically effective amount of an anti-diabetic agent, a hypolipidemic agent, an anti-obesity agent or an anti-hypertensive agent.

Ultimately, the present invention provides a method or use which comprises administering a compound as defined and described above in the form of a pharmaceutical composition as described herein.

As used throughout the specification and in the claims, the term “treatment” embraces all the different forms or modes of treatment as known to those of the pertinent art and in particular includes preventive, curative, delay of progression and palliative treatment.

The above-cited properties are demonstrable in vitro and in vivo tests using advantageously mammals, e.g., mice, rats, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻² molar and 10⁻⁹ molar concentrations. A therapeutically effective amount in vivo may range depending on the route of administration, between about 0.1 mg/kg and 1000 mg/kg, preferably between about 1 mg/kg and 100 mg/kg.

The activity of the DGAT1 inhibitor compound according to the invention may be assessed by the following methods or methods well-described in the art:

The enzyme preparation used in this assay is a membrane preparation from Sf9 cells overexpressing human (His)₆DGAT1. During all steps samples were chilled to 4° C. Sf9 cells expressing human (His)₆DGAT1 were thawed at RT and re-suspended at a 10:1 ratio (mL buffer/g of cells) in 50 mM HEPES, 1× Complete Protease Inhibitor, pH 7.5. The re-suspended pellet was homogenized for 1 min using a Brinkman PT 10/35 homogenizer with a 20 mm generator. Cells were lysed using Avestin Emulsiflex (chilled to 4° C.) at 10000-15000 psi. Lysate was centrifuged at 100,000×g for 1 h at 4° C. Supernatant was removed and pellets were re-suspended in 50 mM HEPES, 1× Complete Protease Inhibitor, pH 7.5 at ⅙ the volume of supernatant. Re-suspended pellets were pooled and homogenized with 10 strokes of a Glas-Col motor driven teflon pestle on setting 70. The protein concentration of the membrane preparation was quantified using BCA protein assay with 1% SDS. The membrane preparation was aliquoted, frozen on dry ice, and stored at −80° C.

For 50 mL, 25 mL of 0.2 M HEPES stock buffer, 0.5 mL of 1 M MgCl₂ (5 mM final concentration), and 24.5 mL of milli-Q H₂O are added to the 55 mL Wheaton Potter-Elvehjem homogenizer. Enzyme preparation (0.1 mL) is added to buffer and the mixture is homogenized with 5 strokes on ice using the Glas-Col variable speed homogenizer system on setting 70.

For 50 mL, 0.5 mL 10 mM diolein is added to 9.5 mL of EtOH in a 50 mL Falcon screw cap conical centrifuge tube. Five mL of 10 mM sodium acetate pH 4.5 is added followed by 0.5 mL of 10 mM oleoyl-CoA. Finally, the remaining 4.5 mL of 10 mM sodium acetate pH 4.5 is added followed by 30 mL of milli-Q H₂O. The solution should be gently agitated by hand to induce mixing. The final concentrations of EtOH and sodium acetate are 20% and 2 mM, respectively.

Dry compounds are dissolved in the appropriate volume of DMSO to a final concentration of 10 mM. A 10-point, 3-fold dose response is used to evaluate compound potency. All dilutions are performed in DMSO in a Greiner 384-well microplate.

1. 2 μL of compound in DMSO is added to the appropriate wells. 2 μL of DMSO is added to 100% activity and 100% inhibition controls.

2. 25 μL of enzyme mix is added to all wells and plate(s) are incubated for 10 min at RT.

3. 10 μL of 20% acetic acid quench is added to 100% inhibition control wells. Plate(s) are vortexed using Troemner multi-tube vortexer (setting 7 for 10 sec).

4. 25 μL of substrate mix is added to all wells. Plate(s) are vortexed using Troemner multi-tube vortexer (setting 7 for 10 sec). Plate(s) are incubated for 30 min at RT.

5. 10 μL of 20% acetic acid quench is added to all wells. Plate(s) are vortexed using Troemner multi-tube vortexer (setting 7 for 10 sec).

6. 50 μL of 1-butanol w/ glyceryl tripalmitoleate internal standard is added to all wells.

7. Plate(s) are sealed with super pierce strong plate sealer using the thermo-sealer.

8. Plate(s) are vortexed using Troemner multi-tube vortexer (setting 10 for 5 min).

9. Plate(s) are centrifuged at 162×g (1000 rpm for GH-3.8 rotor) for 5 min using Beckman GS-6R tabletop centrifuge.

Samples were analyzed by LC/MS/MS using a Waters 1525μ LC and Quattro Micro API MS. Where indicated, tripalmitolein was used as an internal standard to control for instrument variation.

Data is converted to % inhibition prior to curve fitting using the following equation:

$$\% Inhibition = \frac{( \begin{matrix}response compound - \\ response 100 \% inhibition control\end{matrix} )}{( \begin{matrix}response 100 \% activity control - \\ response 100 \% inhibition control\end{matrix} )} \times 100$$

Using the method described above, the DGAT1 inhibitors were shown to possess inhibitory activity with IC50 values ranging from 0.001 uM to 100 uM.

1. A method of treating a disease or condition known as myocardial ischemia, comprising:
administering to a warm-blooded animal in need thereof a therapeutically effective amount of a DGAT1 inhibitor.

2. The method according to claim 1, wherein the warm-blooded animal is a human.

3. (canceled)

4. (canceled)

5. The method of claim 1, wherein the DGAT1 inhibitor is a compound having the following structure
A-L1-B-C-L2-E
and pharmaceutically acceptable salts, and prodrugs thereof, wherein
A is a substituted or unsubstituted alkyl, cycloalkyl, aryl, or heterocyclyl group,
L1 is selected from the group consisting of
an amine group —NH—
a substituted amine group of the formula —N(CH₃)—, —(CH₂NH— or —CH₂—(CH₂NH—,
an amide group —C(O)—NH—,
a sulphonamide group —S(O)₂—NH—, or
a urea group-NHC(O)—NH—,
B is a substituted or unsubstituted, monocyclic, 5 or 6 membered divalent heteroaryl group,
C-D is selected from the following cyclic structures:
C-D together is a substituted or unsubstituted divalent biphenyl group,
C is a substituted or unsubstituted divalent phenyl group and D is a single bond,
C is a substituted or unsubstituted divalent phenyl group, and D is a substituted or unsubstituted divalent non-aromatic monocyclic ring which is selected from a saturated or unsaturated divalent cycloalkyl group or a saturated or unsaturated divalent heterocycloalkyl group,
C-D together is a spiro residue, wherein
the first cyclic component is a benzo-fused cyclic component wherein the ring which is fused to the phenyl part is a 5- or 6 membered ring, optionally comprising one or more heteroatoms, the first cyclic component being attached to the moiety B via its phenyl part, and
the second cyclic component is a cycloalkyl or cycloalkylidenyl residue which is attached to L2,
L2 is selected from the group consisting of:
a single bond,
a divalent residue having the following structure:
—[R¹]ₐ—[R²]_{b}—[C(O)]_{c}—[N(R³)]_{d}—[R⁴]ₑ—[R⁵]_{f}—
wherein
a is 0 or 1,
b is 0 or 1,
c is 0 or 1,
d is 0 or 1,
e is 0 or 1,
f is 0 or 1,
with the proviso that (a+b+c+d+e+f)>0, and c=1 if d=1,
R¹, R², R⁴ and R⁵, which can be the same or different, are a substituted or unsubstituted divalent alkyl, cycloalkyl, alkenyl, alkynyl, alkylene, aryl or heterocycly residue,
R³ is H or hydrocarbyl,
or R³ and R⁴ form together with the nitrogen atom to which they are attached a 5 or 6 membered heterocycloalkyl group,
with the proviso that R¹ and R² are not both alkyl if c=1 and d=e=f=0 and the carbonyl carbon atom is attached to the moiety E,
an alkylidenyl group which is linked to the moiety D via a double bond, and
E is selected from the group consisting of:
a sulphonic acid group and derivatives thereof,
a carboxyl group and derivatives thereof, wherein the carboxyl carbon atom is attached to L2,
a phosphonic acid group and derivatives thereof,
an alpha-keto hydroxyalkyl group,
a hydroxyalkyl group wherein the carbon atom bonded to the hydroxyl group is further substituted with one or two trifluoro-methyl groups,
a substituted or unsubstituted five-membered heterocyclyl residue having in the ring at least two heteroatoms and at least one carbon atom, wherein
the at least one carbon atom of the ring is bonded to two heteroatoms;
at least one of the heteroatoms to which the carbon atom of the ring is bonded is a member of the ring;
and at least one of the heteroatoms to which the carbon atom of the ring is bonded or at least one of the heteroatoms of the ring is bearing a hydrogen atom;
with the provisos that
L2 is not a single bond or a divalent alkyl group if the moiety D is a single bond,
L2 is not a single bond if the moiety D is an unsubstituted divalent phenyl group and E is a carboxylic acid or a derivative thereof,
E is not a carboxamide group if L2 comprises an amide group,
E is not a —COOH group if D is a single bond and L2 is a —N(CH₃)—C(O)— group wherein the carbonyl carbon atom is attached to the moiety E,
L2 is not a divalent N-methyl piperidinyl group if the moiety E is a pyridinyl-1,2,4-triazolyl group,
L2 is not —C(O)-[R⁴]ₑ—[R⁵]_{f}— when C is a substituted or unsubstituted divalent phenyl group and D is a single bond,
or a pharmaceutically acceptable salt thereof.

6. The method of claim 1, wherein the DGAT1 inhibitor is a compound having the following structure
A-L1-B-C-D
and pharmaceutically acceptable salts, and prodrugs thereof,
wherein
A is selected from a substituted or unsubstituted alkyl, substituted or unsubstituted alkoxy, substituted or unsubstituted cycloalkyl, substituted or unsubstituted aryl, and a substituted or unsubstituted heterocycyl, wherein A is linked to L1 via a carbon member of the ring when A is a ring,
L1 is selected from the group consisting of:
an amine group of the formula —(CH₂), CR₄R_{4′})ₚ—(CH₂)ₘ—N(R₃)—,
a thiocarbamoyl group of the formula —(CH₂)ₙ—(CR₄R_{4′})ₚ—(CH₂)ₘ—(R₃)—C(S)—,
an amide group of the formula —C(O)—N(R₃)—(CH₂)ₙ—(CR₄R_{4′})ₚ—(CH₂)ₘ—,
an amidine group of the formula —C(NH)—N(R₃)—(CH₂)ₙ—(CR₄R_{4′})ₚ—(CH₂)ₘ—,
an amide group of the formula —(CH₂)ₙ—(CR₄R_{4′})ₚ—(CH₂)ₘ—C(O)—N(R₃)—,
a sulphonamide group of the formula —(CH₂)ₙ—(CR₄R_{4′})ₚ—(CH₂)ₘ—S(O)₂—N(R₃)—,
a carbamate group of the formula —(CH₂)ₙ—CR₄R_{4′})ₚ—(CH₂)ₘ—(O)—C(O)—N(R₃)—, or
a urea group of the formula —(CH₂)ₙ—(CR₄R_{4′})ₚ—(CH₂)ₘ—N(R₃)—C(O)—N(R_{3A})—,
wherein;
R₃ and R_{3A} are, independently from each other, hydrogen or lower alkyl,
m, n and p are, independently from each other, an integer from 0 to 2,
R₄ and R_{4′} are, independently from each other, hydrogen, halogen, hydroxyl, lower alkoxy, lower alkoxycarbonyl, carboxy or lower alkyl, or R₄ and R_{4′} are joined together to form a spiro residue of the formula 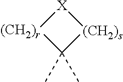
wherein;
X is NR_{3′}, O, S or CR_{3″}R_{4″}
r and s are, independently from each other, zero or an integer from 1 to 3,
R_{3′} is hydrogen or lower alkyl,
R_{3″} is hydrogen, halogen, hydroxyl, alkoxy, or lower alkyl,
R_{4′} is hydrogen or lower alkyl;
B is a substituted or unsubstituted divalent heteroaryl group selected from one of the groups below: 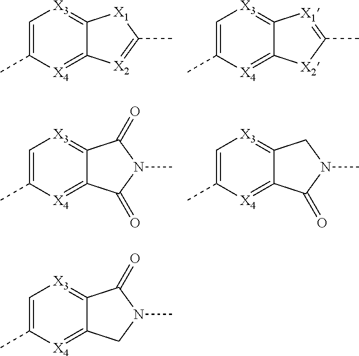
wherein,
X₁ and X₂′ are independently selected from O, NH, NR₉ or S, wherein R₃ is selected from lower alkyl, lower alkylamino, lower alkoxyalkyl, lower hydroxyalkyl,
X₁′, X₂, X₃ and X₄ are independently selected from N, or CH,
C is 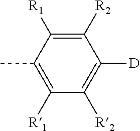
wherein
R₁ is selected from hydrogen, cyano, lower alkylsulfonylamino, alkanoylamino, halogen, lower alkyl, trifluoromethyl, lower alkoxy, lower alkylamino, lower dialkylamino, and NO₂,
R′₁, R₂ and R′₂ are independently selected from hydrogen, halogen, trifluoromethyl, aryloxy, lower alkyl, lower alkoxy, lower alkylamino, lower dialkylamino, and NO₂, or
C may also be a substituted or unsubstituted bicyclic aryl or heteroaryl group,
D is selected from hydrogen, halogen, hydroxyl, cyano, alkanoylamino, carboxy, carbamoyl, —O-L₂-E, —S-L₂-E′, —C(O)— L₂-E, -L₂-E″, and —NR₆-L₂-E′,
L₂ is —(CH₂)ₙ(CR₅R_{5′})ₚ—(CH₂)ₘ—
E is;
alkyl, acyl, alkoxycarbonyl, phosphonic acid, phosphonate, cycloalkoxycarbonyl, aryloxycarbonyl, heterocyclyloxycarbony, carboxy, carbamoyl, sulfonyl, —SO₂—OH, sulfamoyl, sulfonyacarbamoyl, sulfonyloxy, sulfonamido, —C(O)—O—R—PRO, substituted or unsubstituted aryl, substituted or unsubstituted heterocyclyl, or substituted or unsubstituted heteroaryl, and when n′+m′+p′ is equal to zero, E is not sulfonyloxy or sulfonamido,
E′ is;
alkyl, acyl, alkoxycarbonyl, cycloalkoxycarbonyl, aryloxycarbonyl, heterocyclyloxycarbony, carboxy, carbamoyl, sulfonylcarbarnoyl, sulfonyl, —SO₂—OH, sulfamoyl, sulfonamido, phosphonic acid, phosphonate, sulfonyloxy, —C(O)—O—R—PRO, substituted or unsubstituted aryl, substituted or unsubstituted heterocyclyl, or substituted or unsubstituted heteroaryl, and when n′+m′+p′ is equal to zero, E′ is not sulfamoyl, sulfonamido, phosphonic acid, phosphonate, or sulfonyloxy,
E″ is;
alkyl, acyl, alkoxycarbonyl, phosphonic acid, phosphonate, cycloalkoxycarbonyl, aryloxycarbonyl, heterocyclyloxycarbony, carboxy, carbamoyl, sulfonyl, sulfamoyl, sulfonyloxy, sulfonamido, —SO₂—OH, sulfonylcarbamoyl, —C(O)—OR—PRO, substituted or unsubstituted aryl, substituted or unsubstituted heterocycyl, or substituted or unsubstituted heteroaryl,
m′, n′ and p′ are, independently from each other, an integer from 0 to 4,
m′+n′+p′ is between 0 and 12, preferably 0, 1, 2, 3 or 4,
R₅ and R_{5′} are, independently from each other, hydrogen, halogen, hydroxyl, lower alkoxy, or lower alkyl, or R₅ and R_{5′} are joined together to form a spiro residue of the formula 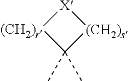
wherein;
X′ is NRₓ, O, S or CR_{x′}R_{x′}
r′ and s′ are, independently from each other, zero or an integer from 1 to 3,
Rₓ is hydrogen or lower alkyl,
Rₓ is hydrogen, halogen, hydroxyl, alkoxy, or lower alkyl,
R_{x′} is hydrogen or lower alkyl; or
a stereoisomer, enantiomer or tautomer thereof, a pharmaceutically acceptable salt thereof, or a prodrug thereof.

7. (canceled)
